(19) United States
(12) Patent Application Publication
(10) Pub. No.: US 2018/0111913 A1
(43) Pub. Date: Apr. 26, 2018

(54) SUBSTITUTED BENZOFURAN DERIVATIVES AS NOVEL ANTIMYCOBACTERIAL AGENTS

(71) Applicant: The Texas A&M University System, College Station, TX (US)

(72) Inventors: James C. Sacchettini, College Station, TX (US); Anup Aggarwal, College Station, TX (US); Maloy K. Parai, College Station, TX (US)

(21) Appl. No.: 15/568,268

(22) PCT Filed: Apr. 22, 2016
(86) PCT No.: PCT/US16/28867

§ 371 Date: Oct. 20, 2017

Related U.S. Application Data
(60) Provisional application No. 62/151,220, filed on Apr. 22, 2015.

(51) Int. Cl. C07D 307/84; C07D 405/04; C07D 235/18; C07D 405/06; A61K 31/4525; A61K 31/4025; A61K 31/55; A61K 31/5377; A61K 31/343; A61K 31/454; A61K 31/496; A61K 31/4545; A61K 45/06; A61P 31/00; A61P 31/06

(52) U.S. Cl. CPC C07D 307/84; A61P 31/06; C07D 235/18; C07D 405/06; A61K 31/4525; A61K 31/4025; A61K 31/55; A61K 31/5377; A61K 31/343; A61K 31/454; A61K 31/496; A61K 31/4545; A61K 45/06; A61P 31/00; C07D 405/04

(57) ABSTRACT

Novel bacterial inhibitors comprising benzofuran derivatives, and methods of bacterial inhibition using the inhibitors are disclosed. The inhibitors may inhibit, for example, mycobacteria, including M. tuberculosis, by inhibition of the Pks13 enzyme. The inhibitors cmat exhibit potent whole cell and in vivo efficacy against M. tuberculosis.

### STATEMENT OF GOVERNMENT INTEREST

The present invention was developed using funding from the National Institutes of Health, Grant No. P01AI095208. The United States government has certain rights in the invention.

### TECHNICAL FIELD

The present disclosure relates to compositions for inhibition of polyketide synthase 13 (“Pks 13”), and for inhibition of pathogenic bacteria expressing Pks 13, including, but not limited to, Mycobacterium tuberculosis (“Mtb”). Certain embodiments of the present disclosure relate to compositions comprising one or more benzofuran derivatives for inhibition of Mtb, and methods of inhibiting Mycobacteria comprising administering such compositions.

### BACKGROUND

### Tuberculosis

Tuberculosis is a common, chronic, and frequently fatal infectious disease caused by various strains of Mycobacteria, most commonly Mtb. Tuberculosis (“TB”) causes more than 1.5 million deaths annually. Emergence of multidrug-resistant Mtb has created an urgent need for the discovery and development of new antitubercular drugs that are effective against the drug-resistant bacteria.

Over twenty drugs are currently used in various combinations for the treatment of TB. These TB drugs are classified into five groups according their effectiveness, potency, drug class and experience of use. First-line TB drugs generally have the greatest activity against TB and include isoniazid, rifampicin, pyrazinamide, ethambutol, rifapentine, and rifabutin. Second-line TB drugs are mainly reserved for the treatment of multidrug-resistant (“MDR”) and X drug-resistant (“XDR”) Mtb. The second-line TB drugs are generally classified into three groups, the first of which includes injectable aminoglycosides (streptomycin, kanamycin, and amikacin) and injectable polypeptides (capreomycin and viomycin), the second group including oral and injectable fluoroquinolones (ciprofloxacin, levofloxacin, moxifloxacin, ofloxacin, and gatifloxacin), and the third including orally administered para-aminosalicylic acid, cycloserine, terizidone, ethionamide, prothionamide, thioacetazone, and linezolid. Third-line anti-TB drugs have unclear efficacy or undefined roles, but they can be tried as last resort drugs. Third-line anti-TB drugs and regimens include clofazimine, linezolid, amoxicillin in combination with clavulanate, imipenem in combination with cilastatin, and clarithromycin.

The current front-line therapy for TB involves a minimum of six months of intensive treatment with a four-drug combination of isoniazid, rifampicin, pyrazinamide, and ethambutol for the treatment of drug-susceptible TB. Treatment of drug-resistant TB can last for up to two years and involves the use of multiple of the second-line drugs, which have severe side effects. The duration and side effects associated with treatment of drug-resistant TB causes significant patient non-compliance, furthering the development of drug resistant Mtb. No single agent exists that is effective in the clinical treatment of tuberculosis, nor is there any combination of agents that offer the possibility of a therapeutic regimen having less than a six month duration. An urgent need exists for novel and potent inhibitors of pathogenic mycobacteria.

### Mycolic Acid Synthesis and Pks13

Mtb has a waxy outer cell-wall layer containing extended long-chain fatty acids called mycolic acids. Mycolic acids are known to be critical for the pathogenicity, virulence, and survival of Mtb. Mycolic acid biosynthesis disruption is therefore an established mechanism of Mtb inhibition, and current front-line antitubercular drugs are known to target this biosynthetic pathway. Isoniazid, for example, targets the enoyl-acyl-ACP reductase enzyme, one of the Fatty Acid Synthase (“FAS”) II system enzymes participating in the elongation of the long-chain (C₄₀-C₆₀) fatty acid components of mycolic acids. However, resistance to isoniazid is increasingly observed in clinical settings and is currently estimated at around 5% worldwide (including monoresistance, as well as MDR and XDR Mtb.

Pks 13 is a large enzyme that catalyzes condensation of two long fatty-acyl chains to synthesize mycolic acids in bacteria of the suborder Corynebacterineae, which includes several important human pathogens, such as Mtb, Mycobacterium leprae, and Corynebacterium diptheriae. In particular, Pks13 performs the final condensation of a long-chain (C₄₀-C₆₀) fatty acid (synthesized by the FAS II system) with a C₂₆ fatty acid (synthesized by the FAS I system) to form an α-hydroxy meromycolate. Pks13 is comprised of five domains that harbor all the activities required for the condensation of two long-chain fatty acids. Pks13 has two acyl carrier protein (ACP) domains, ACP_{N} and ACP_{C}, a β-ketoacyl-synthase (KS), an acyltransferase (AT) and a C-terminal thioesterase (TE) domain. The two ACP domains accept distinct substrates: the ACP_{N} accepts activated meroacyl-AMP from FadD32 and transfers it to the KS domain, and the ACP_{C} is loaded with a 2-carboxyacyl-CoA chain by the AT domain. The KS domain performs the Claisen-type condensation to produce α-alkyl β-ketoester attached to the ACP, which is then released by the TE domain for subsequent modification reactions.

Pks13 has been shown to be essential to Mtb survival and pathogenicity in vitro, and has been presumed to be essential in vivo as well. However, Pks13 is not targeted by any existing drugs in clinical use.

### SUMMARY

The present disclosure relates generally to compositions and methods for inhibiting Pks13 in Corynebacteria, including Mtb in particular. In certain embodiments, the present disclosure provides one or more of: novel benzofuran derivatives; novel inhibitors of Pks13; compositions comprising a Pks13 inhibitor; methods for inhibiting a Corynebacterium; methods of inhibiting Mtb; and methods for inhibiting Pks13 in a pathogenic bacterium. In certain embodiments, the present disclosure provides methods for treating bacterial infections in which the pathogenic bacterium or bacteria express a Pks13 enzyme. The methods can comprise administering a Pks13 inhibitor comprising a benzofuran derivative to a patient infected with a pathogenic bacterium expressing a Pks13 enzyme.

Thus the present disclosure relates, in certain embodiments, to compositions for inhibiting a Pks13 enzyme and/or a bacterium expressing a Pks13 enzyme, including a Corynebacterium such as Mtb, the compositions comprising one or more benzofuran derivatives, pharmaceutically acceptable salts, hydrates, or prodrugs thereof, and combinations thereof (hereinafter, “inhibitors”). In certain embodiments in accordance with the compositions and methods of the present disclosure, the inhibitors comprise benzofuran derivatives, pharmaceutically acceptable salts, hydrates, or prodrugs thereof, and combinations thereof, the derivatives having the general structure:

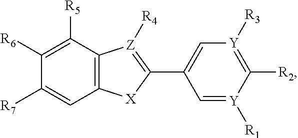

wherein:
X is O or NH;
each Y group is independently selected from C and N;
Z is C or N;
R₁, R₂ and R₃ are independently selected from hydrogen, methoxy, hydroxyl, fluoro, nitrile, and carboxamide moieties;
R₄ is selected from the group consisting of CH₂OH, COOEt, COOH, CONHMe, CONHEt, and amides of cyclic and acyclic secondary or tertiary amines;
R₅ is an alkyl, cyclic alkyl, or heterocyclic alkyl group, optionally substituted with a substituent selected from hydroxyl, alkoxy, halogen, amine, alkylamine, hydroxyalkylamine, dialkylamine, dialkylaminealkyl, carboxy, carboxamide, acylamine, sulfoxide, sulfone, aryl, heteroaryl, and heterocyclic groups (the heretocyclic groups including, for example, morpholine, piperidine, piperizine, pyrrolidine, and azepine groups);
R₆ is selected from H, OMe, and OH; and
R₇ is selected from H, NO₂, NH₂, and NHAc.

According to certain embodiments, the disclosure provides methods of inhibiting a pathogenic bacterium expressing Pks13 by administering one or more inhibitors to the mycobacterium in an amount and for a time sufficient to inhibit the bacterium. According to certain embodiments, the disclosure provides methods of inhibiting a bacterial Pks13 enzyme by administering one or more inhibitors to the bacterium in an amount and for a time sufficient to inhibit the enzyme.

The following abbreviations and shorthand references are used throughout the specification:
Mtb—Mycobacterium tuberculosis
Pks13—Polyketide synthase 13
TE—Thioesterase
FAS—Fatty Acid Synthase
Log—Log₁₀
4-MUH—4-methylumbelliferyl heptanoate
Inhibitor—composition for inhibiting a mycobacterium comprising one or more benzofuran derivatives, pharmaceutically acceptable salts, hydrates, or prodrugs thereof, and combinations thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A more complete understanding of the present embodiments and advantages thereof may be acquired by referring to the following description taken in conjunction with the accompanying drawings, which depict embodiments of the present disclosure, and in which like numbers refer to similar components.

FIG. 1 is a schematic representation of the structure and catalytic site of the Pks13-TE domain showing overall folding and structural elements, with the lid domain (composed of helices α4-α9) shown in cyan and the core domain (comprised of a seven-stranded β-sheet and helices α1-α3, all) shown in orange.

FIG. 2 is a schematic representation of the Pks13-TE domain (orange) superimposed with the E. coli EntF structure (white) showing conserved catalytic residues Ser1533, Aps1560 and His1699, with the hydrogen bonding network between the catalytic residues depicted in dashed lines.

FIG. 3 is a representation of molecular surface structure of the Pks13-TE domain in complex with a first-generation (test) benzofuran derivative, wherein the surface groove in the lid domain is apparent, the benzofuran derivative is shown in stick representation in magenta, the lid domain is shown in cyan, the core domain is shown in orange, and the active site residues Ser1533, Aps1560 and His1699 at the interface of the lid and core domains are shown as ball and stick figures.

FIG. 4 is a schematic representation of the binding interactions of a test benzofuran derivative with the residues from the Pks13-TE lid domain, wherein the benzofuran derivative is rendered in stick form (with C—C bonds in magenta, C—O bonds in red, and C—N bonds in blue), residues of the lid domain that interact with the benzofuran derivative rendered in stick form (with C—C bonds in yellow and C—O and C—N bonds as above), and hydrogen bond interactions are shown as black dashed lines with distances ranging from 2.4 to 3.3 Å.

FIG. 5 is a stereo representation of the test and representative benzofuran derivative compounds C-F bound to the Pks13-TE domain as observed in domain-derivative complex structures, wherein the test derivative is shown in magenta, compound C is shown in green, compound D is shown in yellow, compound E is shown in orange, and compound F is shown in blue, (compounds C, D, and E corresponding to compound IDs 3, 4, and 5, respectively).

FIG. 6 is a graph of in vitro cytotoxicity profile observations of human dermal fibroblast cell growth upon exposure to the test benzofuran derivative compound and compounds B-E, I, and L (corresponding to compound IDs 2-5, 13, and 17, respectively) at various concentrations as shown.

FIG. 7 is a schematic representation of the structure of the wild-type Pks13-TE domain (shown in cyan) in complex with the test benzofuran derivative compound (shown in magenta) superimposed with the structure of the benzofuran derivative-resistance conferring D1607N Pks13-TE mutant domain (shown in yellow), with hydrogen bonding between the test compound and the wild-type Pks13-TE domain represented in dotted lines and altered hydrogen bonding between the test compound and the D1607N Pks13-TE mutant domain represented in dashed lines.

### DESCRIPTION

The present disclosure relates to compositions and methods for inhibition of a bacterium. These compositions and methods are described in further detail below.

Unless otherwise indicated by the specific context of this specification, such bacterium may be any bacterium inhibited by the compositions and methods of the present disclosure. Such bacterium may be a bacterium that expresses Pks13. The bacterium can be of any bacterial species that expresses Pks13, such as any species of the suborder Corynebacteriaea, including any species of the genus Mycobacterium, including Mtb. Furthermore, such bacterium may be a bacterium in a patient. The patient may be any animal. In particular, the patient may be a mammal, such as a human, a pet mammal such as a dog or cat, an agricultural mammal, such as a horse, cow, buffalo, deer, pig, sheep, or goat, or a zoo mammal. Bacterial inhibition, unless otherwise indicated by the specific context of this specification, can include killing the bacterium, such as via apoptosis or necrosis, reducing or arresting the growth of the bacterium, rendering the bacterium more susceptible to the immune system, preventing or reducing bacterial infection, reducing the number of bacteria in a patient, or otherwise negatively affecting a bacterium. The same applies, mutatis mutandis, to a plurality of bacteria of the same or different species.

### Compositions

The compositions of the present disclosure include inhibitor compositions for inhibiting Pks13, and/or inhibiting a pathogenic bacterium expressing Pks13, such as Mtb. The present disclosure further includes inhibitors for use in the treatment of infection by a bacterium expressing Pks13.

Unless specified to the contrary, all reference herein to compositions, inhibitors, benzofuran derivatives, and/or compounds will include any pharmaceutically acceptable salts, hydrates, or prodrugs thereof, and/or combinations thereof. The term “pharmaceutically acceptable salt” refers to salts whose counter ion derives from pharmaceutically acceptable non-toxic acids and bases.

In certain embodiments, the inhibitors comprise one or more compounds represented by the general structure below:

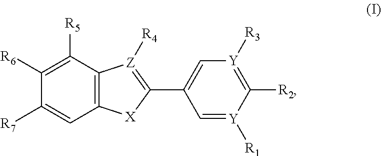

wherein:
X is O or NH;
each Y group is independently selected from C and N;
Z is C or N;
R₁, R₂ and R₃ are independently selected from hydrogen, methoxy, hydroxyl, fluoro, nitrile, and carboxamide moieties;
R₄ is selected from the group consisting of CH₂OH, COOEt, COOH, CONHMe, CONHEt, and amides of cyclic and acyclic secondary or tertiary amines;
R₅ is an alkyl, cyclic alkyl, or heterocyclic alkyl group, optionally substituted with a substituent selected from hydroxyl, alkoxy, halogen, amine, alkylamine, hydroxyalkylamine, dialkylamine, dialkylaminealkyl, carboxy, carboxamide, acylamine, sulfoxide, sulfone, aryl, heteroaryl, and heterocyclic groups (the heretocyclic groups including, for example, morpholine, piperidine, piperizine, pyrrolidine, and azepine groups);
R₆ is selected from H, OMe, and OH; and
R₇ is selected from H, NO₂, NH₂, and NHAc.

Specific compounds of the present disclosure include those having Structure I, as well as those described or characterized elsewhere herein with respect to Structure II, Structure III and Structure 4. Tables 1-7 provide a number of representative compounds of benzofuran derivative Pks13 inhibitors. The minimum inhibitory concentration (“MIC”) and half-maximal inhibitory concentration (“IC₅₀”) of each of the representative inhibitors is also provided. NB indicates no binding; NI indicates no inhibition, and NT indicates not tested Where incomplete inhibition was observed, percentages in square brackets indicate percentage inhibition observed, and concentrations in brackets indicate corresponding concentration.

Significant differences in potency were observed even among close structural analogs. These pharmacokinetic data are discussed in further detail below.

Benzofuran derivatives may also have the following structural formula and the R groups indicated in Table 1:

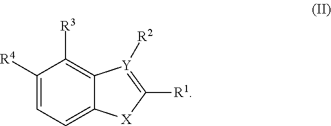

TABLE 1
R Groups for Structure II

| ID | R¹ | R² | R³ | R⁴ | X | Y |
|---|---|---|---|---|---|---|
| 1 | Ph | CO₂Et | 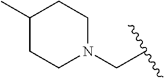 [SMILES: *CN1CCC(C)CC1] | OH | O | C |
| 2 | Ph | CO₂Et | 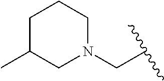 [SMILES: *CN1CCCC(C)C1] | OH | O | C |
| 3 | Ph | CO₂Et | 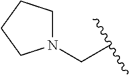 [SMILES: *CN1CCCC1] | OH | O | C |
| 4 | Ph | CO₂Et | 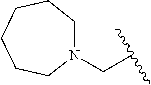 [SMILES: *CN1CCCCCC1] | OH | O | C |
| 5 | Ph | CO₂Et | 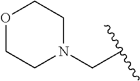 [SMILES: *CN1CCOCC1] | OH | O | C |
| 109 | Ph | CO₂Et | 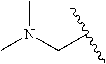 [SMILES: *CN(C)C] | OH | O | C |
| 6 | Ph | 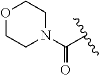 [SMILES: *C(=O)N1CCOCC1] | H | OH | O | C |
| 7 | Ph | CO₂Et | 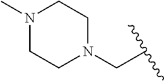 [SMILES: *CN1CCN(C)CC1] | OH | O | C |
| 8 | Ph | | 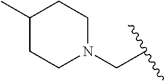 [SMILES: *CN1CCC(C)CC1] | OH | NH | N |
| 9 | CH₃ | | 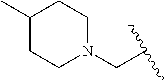 [SMILES: *CN1CCC(C)CC1] | OH | NH | N |
| 110 | Ph | | 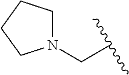 [SMILES: *CN1CCCC1] | OH | NH | N |
| 111 | CH₃ | | 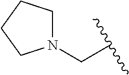 [SMILES: *CN1CCCC1] | OH | NH | N |

Structure II compositions also include the following composition ID 108:

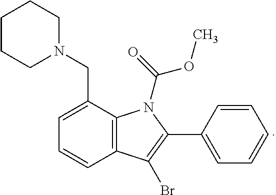 [SMILES: COC(=O)n1c(-c2ccccc2)c(Br)c2cccc(CN3CCCCC3)c21]

TABLE 2
Structure II Pks13 Inhibition Pharmacokinetic Data

| ID | IC₅₀ (μM) | MIC (μM) |
|---|---|---|
| 1 | 0.26 ± 0.03 | 2.3 ± 0.2 |
| 2 | 0.12 ± 0.02 | 4.4 ± 0.2 |
| 3 | 0.24 ± 0.02 | 4.1 |
| 4 | 0.28 ± 0.03 | 4.6 ± 0.3 |
| 5 | 0.71 ± 0.05 | 13.3 ± 1.6 |
| 109 | 1.57 ± 0.15 | 7.3 ± 0.3 |
| 6 | NB | NT |
| 7 | 2.7 | 1.4 |
| 8 | 6.8 | 22 |
| 9 | 10.0 | NI |
| 110 | 6.8 | 22 |
| 111 | 10.0 | NI |
| 108 | 2 | [26%] (20 μM) |

Benzofuran derivatives may also have the following structural formula and the R groups indicated in Table 3:

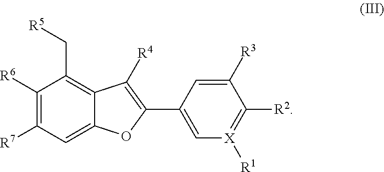

TABLE 3
R Groups for Structure III

| ID | R¹ | R² | R³ | R⁴ | R⁵ | R⁶ | R⁷ | X |
|---|---|---|---|---|---|---|---|---|
| 10 | H | H | H | —CO₂Et | Ph | OH | H | C |
| 12 | H | H | H | —CO₂Et | H | OH | H | C |
| 13 | H | H | H | —CO₂Et | 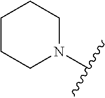 [SMILES: *N1CCCCC1] | OH | H | C |
| 14 | H | H | H | —CO₂H | 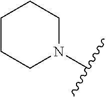 [SMILES: *N1CCCCC1] | OH | H | C |
| 15 | H | H | H | —CO₂Et | 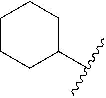 [SMILES: *C1CCCCC1] | OCH₃ | H | C |
| 16 | H | H | H | —CO₂Et | 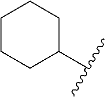 [SMILES: *C1CCCCC1] | OH | H | C |
| 17 | H | H | H | —CONHMe | 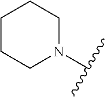 [SMILES: *N1CCCCC1] | OH | H | C |
| 18 | H | H | H | —CONHEt | 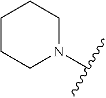 [SMILES: *N1CCCCC1] | OH | H | C |
| 19 | H | H | H | —CO₂Et | Ph | OCH₃ | H | C |
| 21 | H | OCH₃ | H | —CO₂Et | 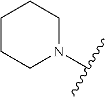 [SMILES: *N1CCCCC1] | OH | H | C |
| 22 | H | OH | H | —CO₂Et | 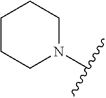 [SMILES: *N1CCCCC1] | OH | H | C |
| 112 | H | H | H | —CONHMe | 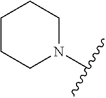 [SMILES: *N1CCCCC1] | OCH₃ | H | N |
| 24 | H | H | H | —CO₂Et | 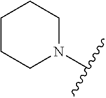 [SMILES: *N1CCCCC1] | H | H | C |
| 25 | H | F | H | —CO₂Et | 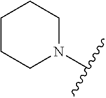 [SMILES: *N1CCCCC1] | OH | H | C |
| 26 | H | H | H | —CO₂Et | 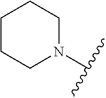 [SMILES: *N1CCCCC1] | OH | NO₂ | C |
| 27 | H | H | H | —CO₂Et | 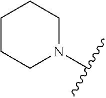 [SMILES: *N1CCCCC1] | OH | NH₂ | C |
| 28 | H | H | H | —CO₂Et | 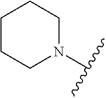 [SMILES: *N1CCCCC1] | OH | NHAc | C |
| 29 | H | F | H | —CONHMe | 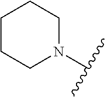 [SMILES: *N1CCCCC1] | OH | H | C |
| 31 | H | OCH₃ | H | —CONHMe | 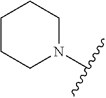 [SMILES: *N1CCCCC1] | OH | H | C |
| 32 | H | OH | H | —CONHMe | 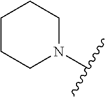 [SMILES: *N1CCCCC1] | OH | H | C |
| 33 | H | H | H | —CONHMe | 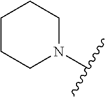 [SMILES: *N1CCCCC1] | H | H | C |
| 34 | H | H | H | —CO₂Et | 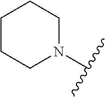 [SMILES: *N1CCCCC1] | H | NH₂ | C |
| 37 | H | H | H | —CO₂Et | 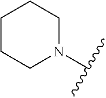 [SMILES: *N1CCCCC1] | H | OCH₃ | C |
| 38 | H | H | H | —CO₂Et | 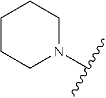 [SMILES: *N1CCCCC1] | H | OH | C |
| 39 | H | H | H | 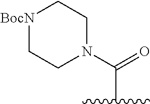 [SMILES: *C(=O)N1CCN(C(=O)OC(C)(C)C)CC1] | 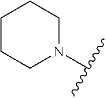 [SMILES: *N1CCCCC1] | OH | H | C |
| 40 | H | H | H | —CONHMe | 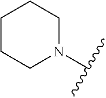 [SMILES: *N1CCCCC1] | H | OH | C |
| 41 | H | H | H | —CONHMe | 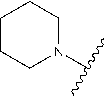 [SMILES: *N1CCCCC1] | OH | H | C |
| 42 | H | H | H | 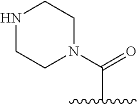 [SMILES: *C(=O)N1CCNCC1] | 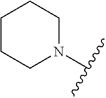 [SMILES: *N1CCCCC1] | OH | H | C |
| 43 | H | H | H | 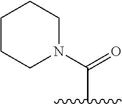 [SMILES: *C(=O)N1CCCCC1] | 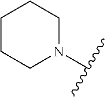 [SMILES: *N1CCCCC1] | OH | H | C |
| 44 | H | H | H | —CONH(CH₂)₂NHAc | 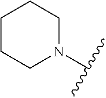 [SMILES: *N1CCCCC1] | OH | H | C |
| 45 | H | OH | H | —CO₂Et | 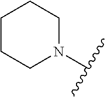 [SMILES: *N1CCCCC1] | H | OH | C |
| 46 | H | H | H | 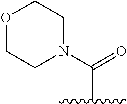 [SMILES: *C(=O)N1CCOCC1] | 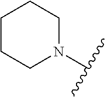 [SMILES: *N1CCCCC1] | OH | H | C |
| 47 | H | OH | H | —CO₂Et | 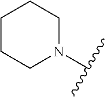 [SMILES: *N1CCCCC1] | H | OH | C |
| 48 | OCH₃ | H | H | —CO₂Et | 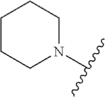 [SMILES: *N1CCCCC1] | OH | H | C |
| 49 | H | F | H | —CO₂Et | 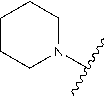 [SMILES: *N1CCCCC1] | H | OH | C |
| 50 | OH | H | H | —CO₂Et | 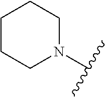 [SMILES: *N1CCCCC1] | OH | H | C |
| 51 | H | F | H | —CO₂Et | 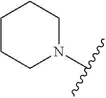 [SMILES: *N1CCCCC1] | H | OH | C |
| 52 | H | H | H | —CO₂Et | 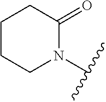 [SMILES: *N1CCCCC1=O] | OH | H | C |
| 53 | H | H | H | 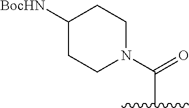 [SMILES: *C(=O)N1CCC(NC(=O)OC(C)(C)C)CC1] | 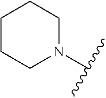 [SMILES: *N1CCCCC1] | OH | H | C |
| 54 | H | H | H | 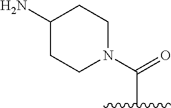 [SMILES: *C(=O)N1CCC(N)CC1] | 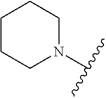 [SMILES: *N1CCCCC1] | OH | H | C |
| 56 | OCH₃ | H | H | —CONHMe | 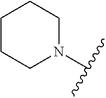 [SMILES: *N1CCCCC1] | OH | H | C |
| 57 | OH | H | H | —CONHMe | 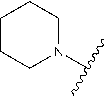 [SMILES: *N1CCCCC1] | OH | H | C |
| 58 | | OH | H | —CO₂Et | 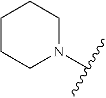 [SMILES: *N1CCCCC1] | OH | H | N |
| 59 | F | OH | H | —CO₂Et | 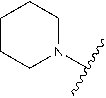 [SMILES: *N1CCCCC1] | OH | H | C |
| 60 | H | CONH₂ | H | —CO₂Et | 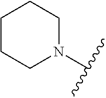 [SMILES: *N1CCCCC1] | OH | H | C |
| 61 | F | OH | H | —CONHMe | 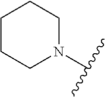 [SMILES: *N1CCCCC1] | OH | H | C |
| 62 | H | CN | H | —CO₂Et | 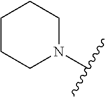 [SMILES: *N1CCCCC1] | OH | H | C |
| 63 | F | OH | F | —CO₂Et | 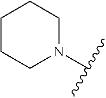 [SMILES: *N1CCCCC1] | OH | H | C |
| 64 | F | OH | F | —CONHMe | 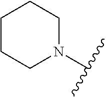 [SMILES: *N1CCCCC1] | OH | H | C |
| 65 | H | H | H | —CH₂OH | 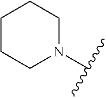 [SMILES: *N1CCCCC1] | OH | H | C |
| 116 | H | H | H | —CO₂Et | 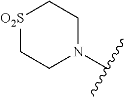 [SMILES: *N1CCS(=O)(=O)CC1] | OH | H | C |
| 117 | H | H | H | —CO₂Et | 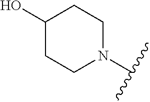 [SMILES: *N1CCC(O)CC1] | OH | H | C |
| 118 | H | OH | H | —CONHMe | 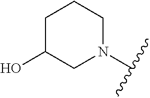 [SMILES: *N1CCCC(O)C1] | OH | H | C |
| 119 | H | H | H | —CO₂Et | 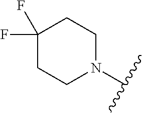 [SMILES: *N1CCC(F)(F)CC1] | OH | H | C |
| 120 | H | OH | H | —CONHMe | 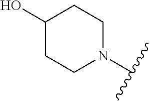 [SMILES: *N1CCC(O)CC1] | OH | H | C |
| 121 | H | OH | H | —CONHMe | 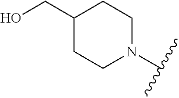 [SMILES: *N1CCC(CO)CC1] | OH | H | C |
| 122 | H | OH | H | —CONHMe | 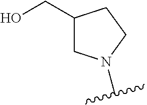 [SMILES: *N1CCC(CO)C1] | OH | H | C |
| 123 | H | OH | H | —CONHMe | 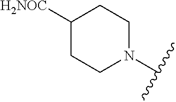 [SMILES: *N1CCC(C(N)=O)CC1] | OH | H | C |
| 124 | H | OH | H | —CONHMe | 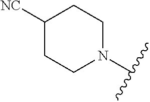 [SMILES: *N1CCC(C#N)CC1] | OH | H | C |
| 125 | H | OH | H | —CONH₂ | 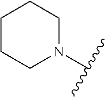 [SMILES: *N1CCCCC1] | OH | H | C |
| 126 | H | OH | H | —CONHMe | 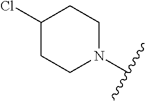 [SMILES: *N1CCC(Cl)CC1] | OH | H | C |
| 127 | H | OH | H | —CONHMe | 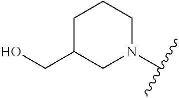 [SMILES: *N1CCCC(CO)C1] | OH | H | C |
| 128 | H | OH | H | —CONHMe | 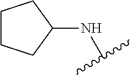 [SMILES: *NC1CCCC1] | OH | H | C |
| 129 | H | OH | H | —CONHMe | 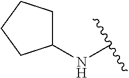 [SMILES: *NC1CCCC1] | H | H | C |
| 130 | H | H | H | —CO₂Et | 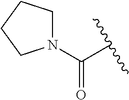 [SMILES: *C(=O)N1CCCC1] | OH | H | C |
| 131 | H | H | H | —CO₂Et | 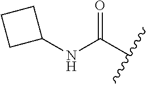 [SMILES: *C(=O)NC1CCC1] | OH | H | C |
| 132 | H | H | H | —CO₂Et | 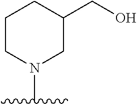 [SMILES: *N1CCCC(CO)C1] | OH | H | C |
| 133 | H | H | H | —CO₂Et | 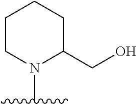 [SMILES: *N1CCCCC1CO] | OH | H | C |
| 134 | H | OH | H | —CO₂Et | 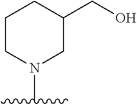 [SMILES: *N1CCCC(CO)C1] | OH | H | C |
| 135 | H | H | H | —CONHMe | 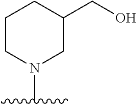 [SMILES: *N1CCCC(CO)C1] | OH | H | C |
| 67 | H | H | H | —CO₂Et | 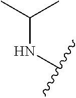 [SMILES: *NC(C)C] | H | OH | C |
| 68 | H | H | H | —CONHMe | 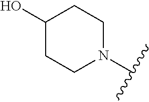 [SMILES: *N1CCC(O)CC1] | H | OH | C |
| 69 | H | H | H | —CO₂Et | 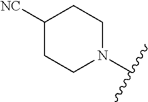 [SMILES: *N1CCC(C#N)CC1] | H | OH | C |
| 70 | H | H | H | —CO₂Et | 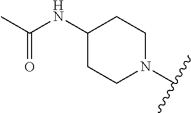 [SMILES: *N1CCC(NC(C)=O)CC1] | H | OH | C |
| 72 | H | H | H | —CO₂Et | 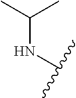 [SMILES: *NC(C)C] | H | OMe | C |

TABLE 4
Other Structure III Compositions

| ID | Structure |
|---|---|
| 11 | 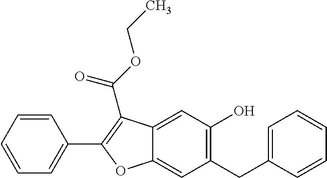 [SMILES: CCOC(=O)c1c(-c2ccccc2)oc2cc(Cc3ccccc3)c(O)cc12] |
| 23 | 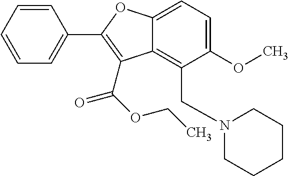 [SMILES: CCOC(=O)c1c(-c2ccccc2)oc2ccc(OC)c(CN3CCCCC3)c12] |
| 35 | 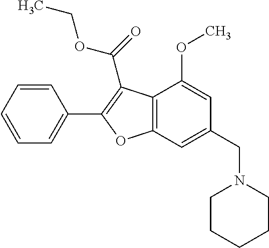 [SMILES: CCOC(=O)c1c(-c2ccccc2)oc2cc(CN3CCCCC3)cc(OC)c12] |
| 55 | 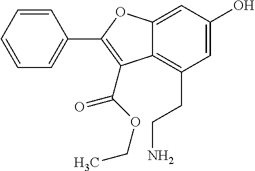 [SMILES: CCOC(=O)c1c(-c2ccccc2)oc2cc(O)cc(CCN)c12] |
| 71 | 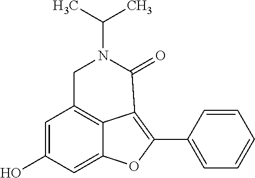 [SMILES: CC(C)N1Cc2cc(O)cc3oc(-c4ccccc4)c(c23)C1=O] |
| 20 | 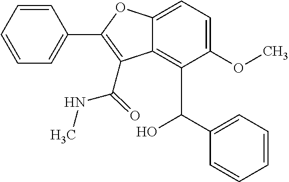 [SMILES: CNC(=O)c1c(-c2ccccc2)oc2ccc(OC)c(C(O)c3ccccc3)c12] |
| 30 | 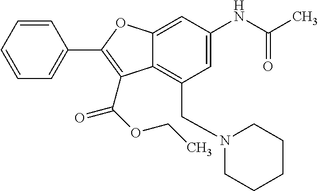 [SMILES: CCOC(=O)c1c(-c2ccccc2)oc2cc(NC(C)=O)cc(CN3CCCCC3)c12] |
| 36 | 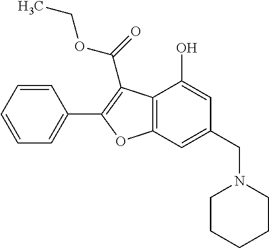 [SMILES: CCOC(=O)c1c(-c2ccccc2)oc2cc(CN3CCCCC3)cc(O)c12] |
| 66 | 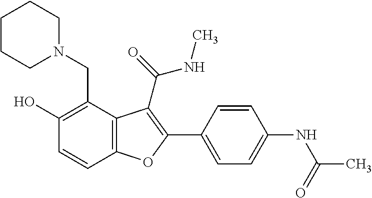 [SMILES: CNC(=O)c1c(-c2ccc(NC(C)=O)cc2)oc2ccc(O)c(CN3CCCCC3)c12] |
| 82 | 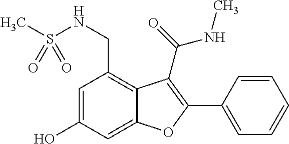 [SMILES: CNC(=O)c1c(-c2ccccc2)oc2cc(O)cc(CNS(C)(=O)=O)c12] |
| 83 | 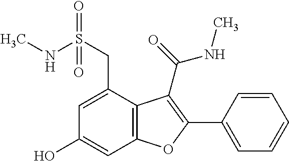 [SMILES: CNC(=O)c1c(-c2ccccc2)oc2cc(O)cc(CS(=O)(=O)NC)c12] |
| 84 | 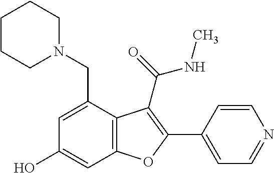 [SMILES: CNC(=O)c1c(-c2ccncc2)oc2cc(O)cc(CN3CCCCC3)c12] |
| 85 | 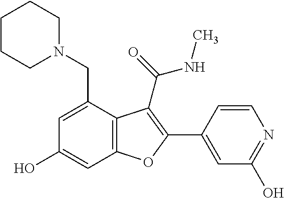 [SMILES: CNC(=O)c1c(-c2ccnc(O)c2)oc2cc(O)cc(CN3CCCCC3)c12] |
| 87 | 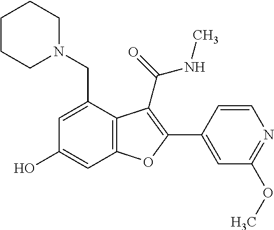 [SMILES: CNC(=O)c1c(-c2ccnc(OC)c2)oc2cc(O)cc(CN3CCCCC3)c12] |
| 89 | 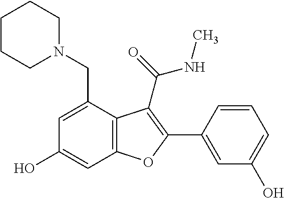 [SMILES: CNC(=O)c1c(-c2cccc(O)c2)oc2cc(O)cc(CN3CCCCC3)c12] |
| 91 | 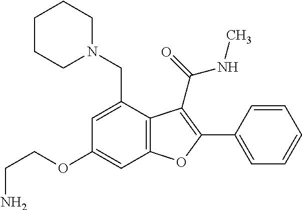 [SMILES: CNC(=O)c1c(-c2ccccc2)oc2cc(OCCN)cc(CN3CCCCC3)c12] |
| 93 | 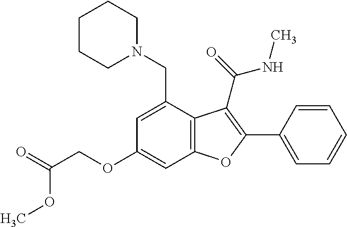 [SMILES: CNC(=O)c1c(-c2ccccc2)oc2cc(OCC(=O)OC)cc(CN3CCCCC3)c12] |
| 86 | 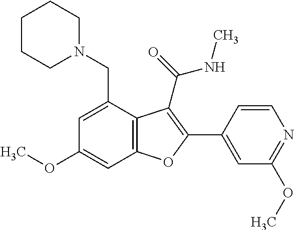 [SMILES: CNC(=O)c1c(-c2ccnc(OC)c2)oc2cc(OC)cc(CN3CCCCC3)c12] |
| 88 | 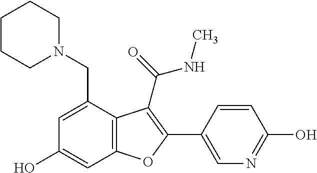 [SMILES: CNC(=O)c1c(-c2ccc(O)nc2)oc2cc(O)cc(CN3CCCCC3)c12] |
| 90 | 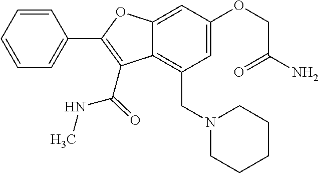 [SMILES: CNC(=O)c1c(-c2ccccc2)oc2cc(OCC(N)=O)cc(CN3CCCCC3)c12] |
| 92 | 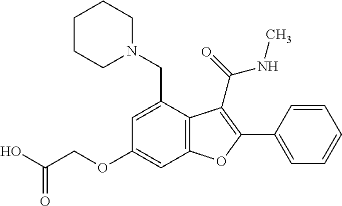 [SMILES: CNC(=O)c1c(-c2ccccc2)oc2cc(OCC(=O)O)cc(CN3CCCCC3)c12] |
| 94 | 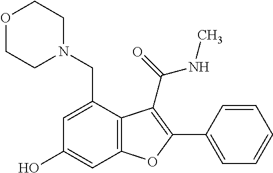 [SMILES: CNC(=O)c1c(-c2ccccc2)oc2cc(O)cc(CN3CCOCC3)c12] |
| 95 | 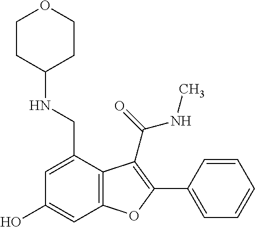 [SMILES: CNC(=O)c1c(-c2ccccc2)oc2cc(O)cc(CNC3CCOCC3)c12] |
| 96 | 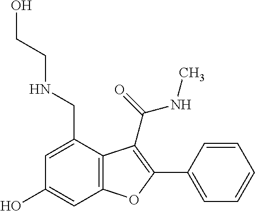 [SMILES: CNC(=O)c1c(-c2ccccc2)oc2cc(O)cc(CNCCO)c12] |
| 97 | 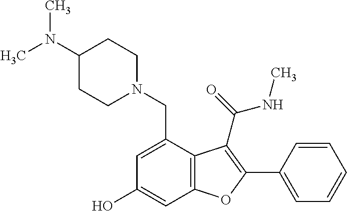 [SMILES: CNC(=O)c1c(-c2ccccc2)oc2cc(O)cc(CN3CCC(N(C)C)CC3)c12] |
| 99 | 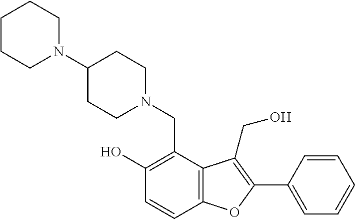 [SMILES: OCc1c(-c2ccccc2)oc2ccc(O)c(CN3CCC(N4CCCCC4)CC3)c12] |
| 101 | 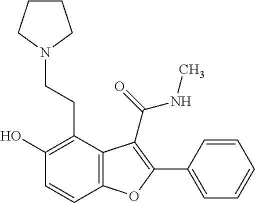 [SMILES: CNC(=O)c1c(-c2ccccc2)oc2ccc(O)c(CCN3CCCC3)c12] |
| 103 | 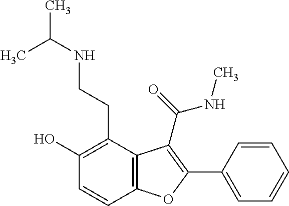 [SMILES: CNC(=O)c1c(-c2ccccc2)oc2ccc(O)c(CCNC(C)C)c12] |
| 105 | 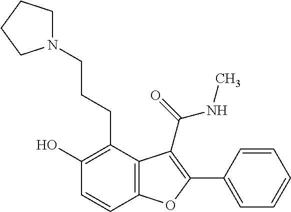 [SMILES: CNC(=O)c1c(-c2ccccc2)oc2ccc(O)c(CCCN3CCCC3)c12] |
| 98 | 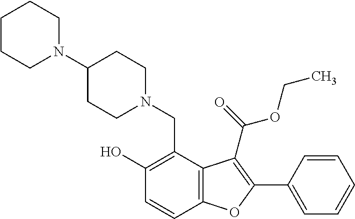 [SMILES: CCOC(=O)c1c(-c2ccccc2)oc2ccc(O)c(CN3CCC(N4CCCCC4)CC3)c12] |
| 100 | 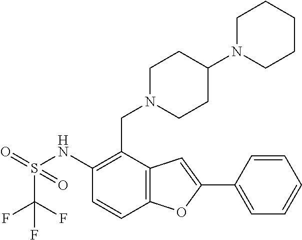 [SMILES: O=S(=O)(Nc1ccc2oc(-c3ccccc3)cc2c1CN1CCC(N2CCCCC2)CC1)C(F)(F)F] |
| 102 | 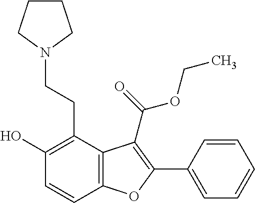 [SMILES: CCOC(=O)c1c(-c2ccccc2)oc2ccc(O)c(CCN3CCCC3)c12] |
| 104 | 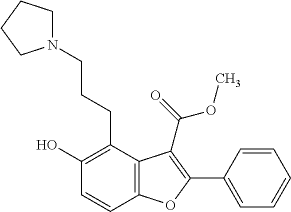 [SMILES: COC(=O)c1c(-c2ccccc2)oc2ccc(O)c(CCCN3CCCC3)c12] |
| 106 | 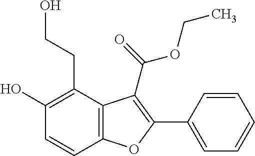 [SMILES: CCOC(=O)c1c(-c2ccccc2)oc2ccc(O)c(CCO)c12] |
| 107 | 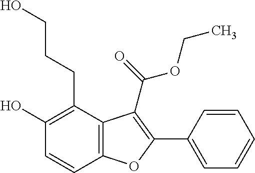 [SMILES: CCOC(=O)c1c(-c2ccccc2)oc2ccc(O)c(CCCO)c12] |

TABLE 5
Structure III Pks13 Inhibition Pharmacokinetic Data

| ID | IC₅₀ (μM) | MIC (μM) | ID | IC₅₀ (μM) | MIC (μM) | ID | IC₅₀ (μM) | MIC (μM) |
|---|---|---|---|---|---|---|---|---|
| 10 | 11.9 | 40 | 25 | 0.35 | 1.5 | 41 | 0.44 | 20 |
| 12 | >20 | 20 | 26 | 1.3 | 5.4 | 42 | 0.74 | 3.1 |
| 13 | 0.26 | 0.4 | 27 | 0.37 | 8 | 43 | 0.32 | >10 |
| 14 | 6.6 | NI | 28 | 3.1 | 20 | 44 | 0.5 | >40 |
| 15 | NB | NI | 29 | 0.3 | 4.5 | 45 | 0.44 | 1.1 |
| 16 | 19.6 | 5.2 | 31 | 0.26 | 1.1 | 46 | 0.34 | >20 |
| 17 | 0.29 | 2 | 32 | 0.19 | 0.09 | 47 | 0.57 | 1.0 |
| 18 | 0.36 | 14 | 33 | 45 | >50 | 48 | 0.36 | 1.3 |
| 19 | 50.0 | 50 | 34 | 4.5 | 9.2 | 49 | 0.4 | >10 |
| 21 | 0.29 | 2.6 | 37 | 29 | 7.3 | 50 | 0.23 | 0.23 |
| 22 | 0.19 | 0.6 | 38 | 0.45 | 6.7 | 51 | 0.8 | >30 |
| 112 | 35.8 | >40 | 39 | 0.66 | 2.1 | 52 | >20 | NT |
| 24 | 2.0 | 16 | 40 | 0.6 | 20 | 53 | 0.63 | 6.6 |
| 54 | 5.3 | 20 | 119 | 0.8 | NA | 133 | >10 | NT |
| 56 | 0.4 | 9.0 | 120 | 0.5 | 4.2 | 134 | 0.4 | 2.5 |
| 57 | 0.27 | 0.44 | 121 | 0.6 | 40 | 135 | 2.0 | 0.5 |
| 58 | 0.36 | 4.0 | 122 | 0.51 | 1.0 | 67 | 0.70 | 9.0 |
| 59 | 0.29 | 0.3 | 123 | 1.6 | >40 | 68 | >20 | NT |
| 60 | 0.3 | 3.5 | 124 | 0.25 | 20 | 69 | 1.6 | 20 |
| 61 | 0.33 | 0.5 | 125 | 0.17 | 1.3 | 70 | 6.1 | >40 |
| 62 | 0.27 | 1.5 | 126 | NA | NA | 72 | >10 | 7.4 |
| 63 | 0.5 | 7.0 | 127 | 1.1 | 5.0 | 11 | NT | 28 |
| 64 | 1.5 | >40 | 128 | 1.0 | 1.0 | 23 | NT | 35.8 |
| 65 | 0.24 | 12.5 | 129 | NA | NA | 35 | NT | 40 |
| 116 | NA | NA | 130 | NA | 4.1 | 55 | 4 | >20 |
| 117 | 0.18 | 5.6 | 131 | NA | 7.2 | 71 | 9 | >20 |
| 118 | 0.48 | 5.0 | 132 | 0.42 | 10 | 20 | 50 | 50 |
| 30 | NT | 20-27 | 93 | 10 | [16%] (40 μM) | 101 | 10 | 3.3 |
| 36 | NT | 40 | 86 | >20 | >20 | 103 | >20 | 0.45 |
| 66 | NT | >20 | 88 | NT | 2.8 | 105 | 5 | 5 |
| 82 | NT | NI | 90 | [31%] (40 μM) | [13%] (20 μM) | 98 | >20 | [40%] (20 μM) |
| 83 | NT | >20 | 92 | INS | INS | 100 | >40 | [6%] (20 μM) |
| 84 | NT | 6.1 | 94 | NT | >20 | 102 | 2.5 | 0.7 |
| 85 | NT | >10 | 95 | NT | 8 | 104 | 2 | 1.8 |
| 87 | NT | 3.3 | 96 | NT | 9 | 106 | NT | >20 |
| 89 | 9 | 1.2 | 97 | NT | NI | 107 | NT | >20 |
| 91 | 10 | [34%] (40 μM) | 99 | NI | [28%] (20 μM) | | | |

Benzofuran derivatives may also have the following structural formula and the R groups indicated in Table 6:

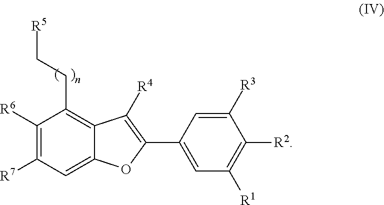

TABLE 6
R Groups for Structure IV

| ID | R¹ | R² | R³ | R⁴ | R⁵ | R⁶ | R⁷ | n | |
|---|---|---|---|---|---|---|---|---|---|
| 136 | H | H | H | —CO₂Et | NH₂ | H | OH | 1 | |
| 137 | H | H | H | —CONHMe | 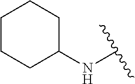 [SMILES: *NC1CCCCC1] | OH | H | 1 | |
| 138 | H | H | H | —CONHMe | 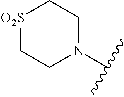 [SMILES: *N1CCS(=O)(=O)CC1] | OH | H | 1 | |
| 139 | H | H | H | —CONHMe | 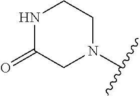 [SMILES: *N1CCNC(=O)C1] | OH | H | 1 | |
| 140 | H | H | H | —CONHMe | 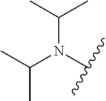 [SMILES: *N(C(C)C)C(C)C] | OH | H | 1 | |
| 141 | H | H | H | —CONHMe | 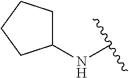 [SMILES: *NC1CCCC1] | OH | H | 1 | |
| 142 | H | H | H | —CONHMe | 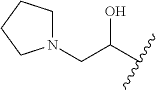 [SMILES: *C(O)CN1CCCC1] | OH | H | 1 | |
| 143 | H | H | H | —CONHMe | 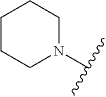 [SMILES: *N1CCCCC1] | OH | H | 1 | |
| 144 | H | H | H | —CONHMe | 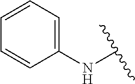 [SMILES: *Nc1ccccc1] | OH | H | 1 | |
| 145 | H | H | H | —CONHMe | 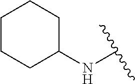 [SMILES: *NC1CCCCC1] | OH | H | 1 | |
| 146 | H | H | H | —CONHMe | 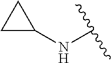 [SMILES: *NC1CC1] | OH | H | 1 | |
| 147 | H | H | H | —CONHMe | 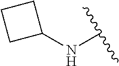 [SMILES: *NC1CCC1] | OH | H | 1 | |
| 148 | H | H | H | —CO₂H | 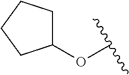 [SMILES: *OC1CCCC1] | OH | H | 1 | |
| 149 | H | H | H | —CO₂Et | 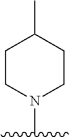 [SMILES: *N1CCC(C)CC1] | OH | H | 1 | |
| 150 | H | H | H | —CO₂Et | 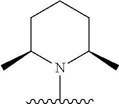 | OH | H | 1 | |
| 151 | H | H | H | —CO₂Et | 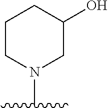 [SMILES: *N1CCCC(O)C1] | OH | H | 1 | |
| 152 | H | H | H | —CONHMe | 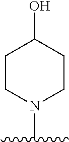 [SMILES: *N1CCC(O)CC1] | OH | H | 1 | |
| 153 | H | H | H | —CONHMe | 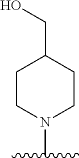 [SMILES: *N1CCC(CO)CC1] | OH | H | 1 | |
| 154 | H | H | H | —CONHMe | 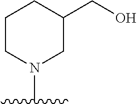 [SMILES: *N1CCCC(CO)C1] | OH | H | 1 | |
| 155 | H | H | H | —CONHMe | 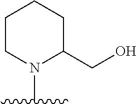 [SMILES: *N1CCCCC1CO] | OH | H | 1 | |
| 156 | H | H | H | —CONHMe | 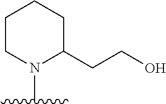 [SMILES: *N1CCCCC1CCO] | OH | H | 1 | |
| 157 | H | H | H | —CONHMe | 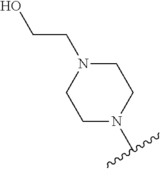 [SMILES: *N1CCN(CCO)CC1] | OH | H | 1 | |
| 158 | H | H | H | —CONHMe | 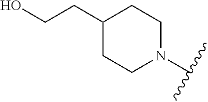 [SMILES: *N1CCC(CCO)CC1] | OH | H | 1 | |
| 159 | H | H | H | —CONHMe | 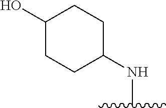 [SMILES: *NC1CCC(O)CC1] | OH | H | 1 | |
| 160 | H | H | H | —CONHMe | 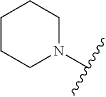 [SMILES: *N1CCCCC1] | OH | H | 2 | |
| 161 | H | H | H | —CO2Et | 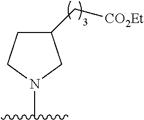 | OH | H | 2 | |
| 162 | H | H | H | —CO₂Et | 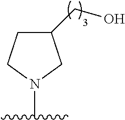 | OH | H | 2 | |
| 163 | H | H | H | —CONHMe | 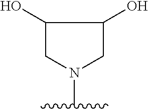 [SMILES: *N1CC(O)C(O)C1] | OH | H | 2 | |
| 164 | H | H | H | —CO₂Et | 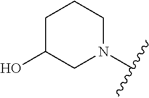 [SMILES: *N1CCCC(O)C1] | OH | H | 2 | |
| 165 | H | H | H | —CO₂Et | 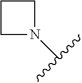 [SMILES: *N1CCC1] | OH | H | 2 | |
| 166 | H | H | H | —CO₂Et | 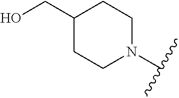 [SMILES: *N1CCC(CO)CC1] | OH | H | 2 | |
| 167 | H | H | H | —CONHMe | 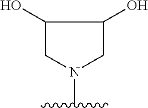 [SMILES: *N1CC(O)C(O)C1] | OH | H | 1 | |
| 168 | H | H | H | —CONHMe | 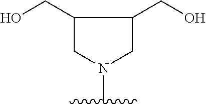 [SMILES: *N1CC(CO)C(CO)C1] | OH | H | 1 | |
| 169 | H | H | H | —CO₂Et | 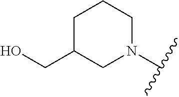 [SMILES: *N1CCCC(CO)C1] | OH | H | 2 | |
| 170 | H | H | H | —CO₂Et | 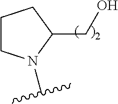 | OH | H | 2 | |
| 171 | H | H | H | —CO₂Et | 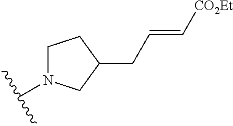 [SMILES: *N1CCC(C/C=C/C(=O)OCC)C1] | OH | H | 2 | |
| 172 | H | H | H | —CO₂Et | 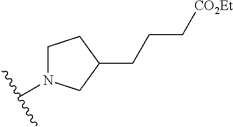 [SMILES: *N1CCC(CCCC(=O)OCC)C1] | OH | H | 2 | |
| 173 | H | H | H | —CO₂Et | 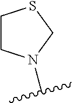 [SMILES: *N1CCSC1] | OH | H | 2 | |
| 174 | H | H | H | —CO₂Et | 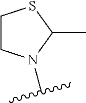 [SMILES: *N1CCSC1C] | OH | H | 2 | |
| 175 | H | H | H | —CO₂Et | 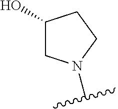 [SMILES: *N1CC[C@@H](O)C1] | OH | H | 2 | |
| 176 | H | H | H | —CO₂Et | 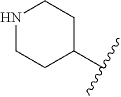 [SMILES: *C1CCNCC1] | OH | H | 1 | |
| 177 | H | H | H | —CO₂Et | 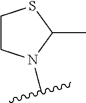 [SMILES: *N1CCSC1C] | OH | H | 1 | |
| 178 | H | H | H | —CO₂Et | 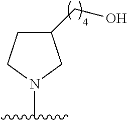 | OH | H | 1 | |
| 179 | H | H | H | —CO₂Et | 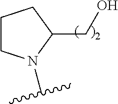 | OH | H | 1 | |
| 180 | H | H | H | —CO₂Et | 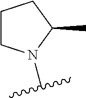 [SMILES: *N1CCC[C@H]1C] | OH | H | 2 | |
| 181 | H | H | H | —CO₂Et | 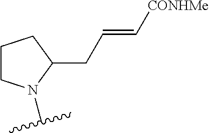 | OH | H | 2 | |
| 182 | H | H | H | —CONHMe | 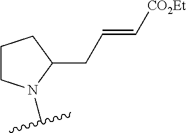 [SMILES: *N1CCCC1C/C=C/C(=O)OCC] | H | OH | 2 | |
| 183 | H | H | H | —CO₂Et | 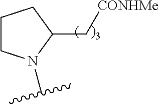 | OH | H | 2 | |
| 184 | H | H | H | —CO₂Et | 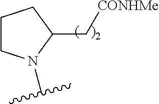 | OH | H | 2 | |
| 185 | H | H | H | —CO₂Et | 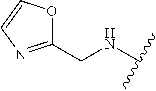 [SMILES: *NCc1ncco1] | OH | H | 1 | |
| 186 | H | H | H | —CO₂Et | 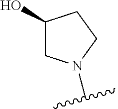 [SMILES: *N1CC[C@H](O)C1] | OH | H | 2 | |
| 187 | H | H | H | —CO₂Et | 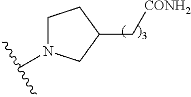 | OH | H | 1 | |
| 188 | H | H | H | —CO₂Et | 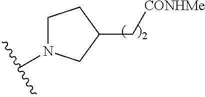 | OH | H | 1 | |
| 189 | H | H | H | —CO₂Et | 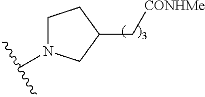 | OH | H | 2 | |
| 190 | H | H | H | —CONHMe | 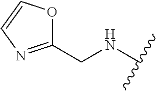 [SMILES: *NCc1ncco1] | OH | H | 2 | |
| 191 | H | H | H | —CO₂Et | 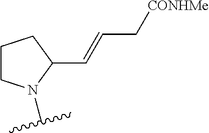 | OH | H | 2 | |
| 192 | H | H | H | —CO₂Et | 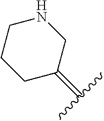 [SMILES: *=C1CCCNC1] | OH | H | 1 | |
| 193 | H | H | H | —CO₂Et | 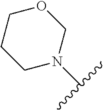 [SMILES: *N1CCCOC1] | OH | H | 1 | |
| 194 | H | H | H | —CONHMe | 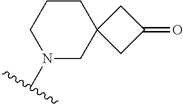 [SMILES: *N1CCCC2(CC(=O)C2)C1] | OH | H | 2 | |
| 195 | H | H | H | —CONHMe | 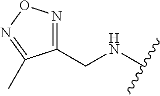 [SMILES: *NCc1nonc1C] | OH | H | 2 | |
| 196 | H | H | H | —CONHMe | 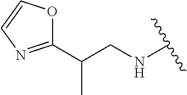 [SMILES: *NCC(C)c1ncco1] | OH | H | 2 | |
| 197 | H | H | H | —CO₂Et | 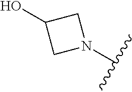 [SMILES: *N1CC(O)C1] | OH | H | 2 | |
| 198 | H | H | H | —CO₂Et | 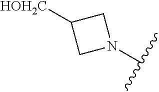 [SMILES: *N1CC(CO)C1] | OH | H | 2 | |
| 199 | H | H | H | —CO₂Et | 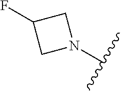 [SMILES: *N1CC(F)C1] | OH | H | 2 | |
| 200 | H | H | H | —CONHMe | 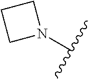 [SMILES: *N1CCC1] | OH | H | 2 | |
| 201 | H | OH | H | —CO₂Et | 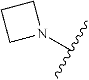 [SMILES: *N1CCC1] | OH | H | 2 | |
| 202 | H | OH | H | —CONHMe | 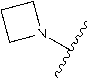 [SMILES: *N1CCC1] | OH | H | 2 | |
| 203 | H | H | H | —CONHMe | 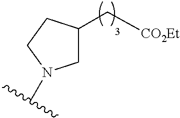 | OH | H | 2 | |
| 204 | H | OH | H | —CONHMe | 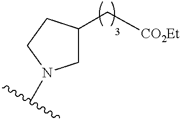 | OH | H | 2 | |
| 205 | H | H | H | —CONHMe | 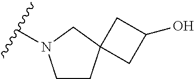 [SMILES: *N1CCC2(CC(O)C2)C1] | OH | H | 2 | |
| 206 | H | H | H | —CONHMe | 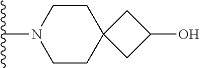 [SMILES: *N1CCC2(CC1)CC(O)C2] | OH | H | 2 | |
| 207 | H | H | H | —CONHMe | 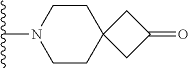 [SMILES: *N1CCC2(CC1)CC(=O)C2] | OH | H | 2 | |
| 208 | H | OH | H | —CONMe₂ | 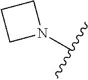 [SMILES: *N1CCC1] | H | OH | 2 | |
| 209 | H | OH | H | —CONMe₂ | 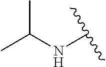 [SMILES: *NC(C)C] | OH | H | 1 | |
| 210 | H | OH | H | —CONMe₂ | 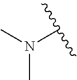 [SMILES: *N(C)C] | OH | H | 1 | |
| 211 | H | H | H | —CONHⁱPrMe | 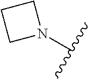 [SMILES: *N1CCC1] | OH | H | 2 | |
| 212 | H | H | H | —CO₂Et | 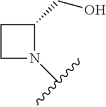 [SMILES: *N1CC[C@@H]1CO] | OH | H | 2 | |
| 213 | H | H | H | —CO₂Et | 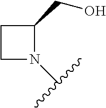 [SMILES: *N1CC[C@H]1CO] | OH | H | 2 | |
| 214 | H | OH | H | —CONHⁱPr | 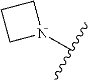 [SMILES: *N1CCC1] | OH | H | 2 | |
| 215 | H | H | H | —CO₂Et | 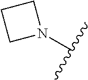 [SMILES: *N1CCC1] | OH | H | 3 | |
| 216 | H | H | H | —CO₂H | 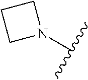 [SMILES: *N1CCC1] | OH | H | 2 | |
| 217 | H | OH | H | 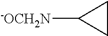 | 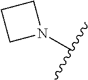 [SMILES: *N1CCC1] | OH | H | 2 | |
| 218 | H | H | H | —CO₂Et | 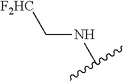 [SMILES: *NCC(F)F] | OH | H | 2 | |
| 219 | H | H | H | —CO₂Et | 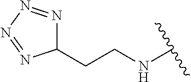 [SMILES: *NCCC1N=NN=N1] | OH | H | 2 | |
| 220 | H | H | H | —CO₂Et | 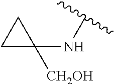 [SMILES: *NC1(CO)CC1] | OH | H | 1 | |
| 221 | H | H | H | —CO₂Et | 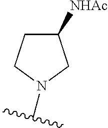 [SMILES: *N1CC[C@@H](NC(C)=O)C1] | OH | H | 1 | |
| 222 | H | OH | H | —CONHMe | 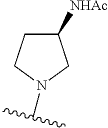 [SMILES: *N1CC[C@@H](NC(C)=O)C1] | OH | H | 2 | |
| 223 | H | OH | H | —CONHEt | 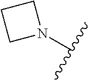 [SMILES: *N1CCC1] | OH | H | 2 | |
| 224 | H | OH | H | —CONHCH₂CF₃ | 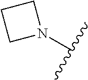 [SMILES: *N1CCC1] | OH | H | 2 | |
| 73 | H | F | H | —CO₂Et | 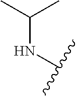 [SMILES: *NC(C)C] | H | OH | 1 | |
| 74 | H | F | H | —CO₂Et | 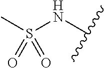 [SMILES: *NS(C)(=O)=O] | H | OH | 1 | |
| 75 | H | F | H | —CO₂Et | 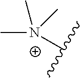 [SMILES: *[N+](C)(C)C] | H | OH | 1 | |
| 76 | H | F | H | —CH₂OH | 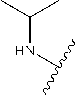 [SMILES: *NC(C)C] | H | OH | 1 | |
| 77 | H | F | H | —CO₂Et | 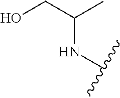 [SMILES: *NC(C)CO] | H | OH | 1 | |
| 78 | H | F | H | —CO₂Et | 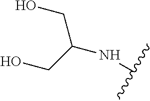 [SMILES: *NC(CO)CO] | H | OH | 1 | |
| 79 | H | F | H | —CO₂Et | 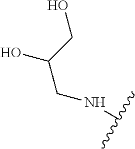 [SMILES: *NCC(O)CO] | H | OH | 1 | |
| 80 | H | F | H | —CO₂Et | 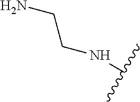 [SMILES: *NCCN] | H | OH | 1 | |
| 81 | H | F | H | —CO₂Et | 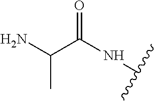 [SMILES: *NC(=O)C(C)N] | H | OH | 1 | |
| 225 | H | F | H | —CO₂Et | 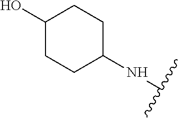 [SMILES: *NC1CCC(O)CC1] | H | OH | 1 | |
| 226 | H | F | H | —CO₂Et | 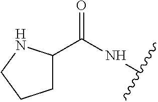 [SMILES: *NC(=O)C1CCCN1] | H | OH | 1 | |
| 227 | H | H | H | —CO₂Et | 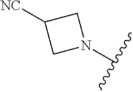 [SMILES: *N1CC(C#N)C1] | OH | H | 1 | |
| 228 | H | H | H | —CO₂Et | 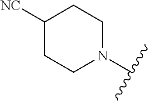 [SMILES: *N1CCC(C#N)CC1] | OH | H | 1 | |
| 229 | H | H | H | —CO₂Et | 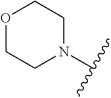 [SMILES: *N1CCOCC1] | OH | H | 1 | |
| 230 | H | H | H | —CONHMe | 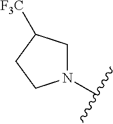 [SMILES: *N1CCC(C(F)(F)F)C1] | OH | H | 1 | |
| 231 | H | H | H | —CONHMe | 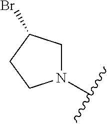 [SMILES: *N1CC[C@H](Br)C1] | OH | H | 1 | |
| 232 | H | H | H | —CONHMe | 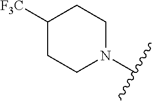 [SMILES: *N1CCC(C(F)(F)F)CC1] | OH | H | 1 | |
| 233 | H | H | H | —CONHMe | 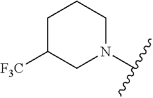 [SMILES: *N1CCCC(C(F)(F)F)C1] | OH | H | 1 | |
| 234 | H | H | H | —CONHMe | 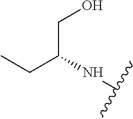 [SMILES: *N[C@H](CC)CO] | OH | H | 1 | |
| 235 | H | H | H | —CONHMe | 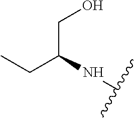 [SMILES: *N[C@@H](CC)CO] | OH | H | 1 | |
| 236 | H | H | H | —CO₂Et | 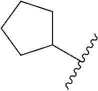 [SMILES: *C1CCCC1] | OH | H | 2 | |
| 237 | H | H | H | —CONHMe | 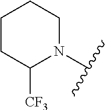 [SMILES: *N1CCCCC1C(F)(F)F] | OH | H | 1 | |
| 238 | H | H | H | —CONHMe | 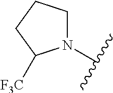 [SMILES: *N1CCCC1C(F)(F)F] | OH | H | 1 | |
| 239 | H | H | H | —CONHMe | 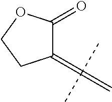 | OH | H | 1 | |
| 240 | H | H | H | —CONHMe | 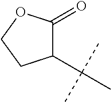 | OH | H | 1 | |
| 241 | H | H | H | —CO₂Et | 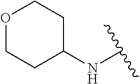 [SMILES: *NC1CCOCC1] | OH | H | 1 | |
| 242 | H | F | H | —CO₂Et | NH₂ | H | OH | 1 | |
| 243 | H | F | H | —CO₂Et | 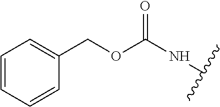 [SMILES: *NC(=O)OCc1ccccc1] | H | OH | 1 | |
| 244 | H | H | H | —CO₂Et | 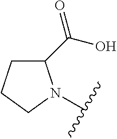 [SMILES: *N1CCCC1C(=O)O] | OH | H | 1 | |
| 245 | H | H | H | —CO₂Et | 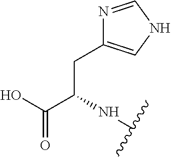 [SMILES: *N[C@@H](Cc1c[nH]cn1)C(=O)O] | OH | H | 1 | |
| 246 | H | H | H | —CO₂Et | 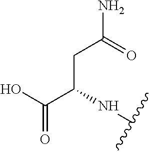 [SMILES: *N[C@@H](CC(N)=O)C(=O)O] | OH | H | 1 | |
| 247 | H | H | H | —CO₂Et | 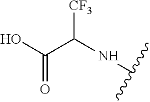 [SMILES: *NC(C(=O)O)C(F)(F)F] | OH | H | 1 | |
| 248 | H | H | H | —CO₂Et | 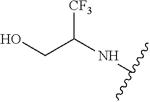 [SMILES: *NC(CO)C(F)(F)F] | OH | H | 1 | |
| 249 | H | H | H | —CO₂Et | 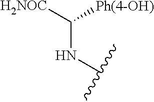 | OH | H | 1 | |
| 250 | H | H | H | —CONHMe | 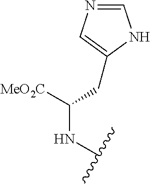 [SMILES: *N[C@@H](Cc1cnc[nH]1)C(=O)OC] | OH | H | 1 | |
| 251 | H | H | H | —CONHMe | 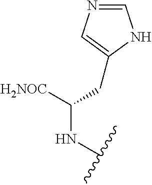 [SMILES: *N[C@@H](Cc1cnc[nH]1)C(N)=O] | OH | H | 1 | |
| 252 | H | H | H | —CONHMe | 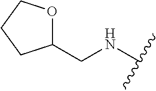 [SMILES: *NCC1CCCO1] | OH | H | 1 | |
| 253 | H | H | H | —CONHMe | 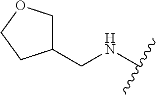 [SMILES: *NCC1CCOC1] | OH | H | 1 | |
| 254 | H | H | H | —CO₂Et | 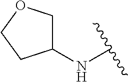 [SMILES: *NC1CCOC1] | OH | H | 1 | |
| 255 | H | H | H | —CO₂Et | 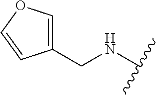 [SMILES: *NCc1ccoc1] | OH | H | 2 | |
| 256 | H | H | H | —CO₂Et | 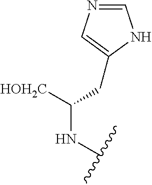 [SMILES: *N[C@H](CO)Cc1cnc[nH]1] | OH | H | 1 | |
| 257 | H | H | H | —CONHMe | 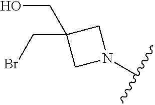 [SMILES: *N1CC(CO)(CBr)C1] | OH | H | 1 | |
| 258 | H | H | H | —CONHMe | 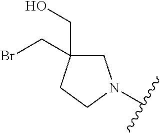 [SMILES: *N1CCC(CO)(CBr)C1] | OH | H | 1 | |
| 259 | H | H | H | —CONHMe | 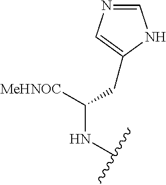 | OH | H | 1 | |
| 260 | H | H | H | —CONHMe | 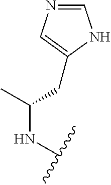 [SMILES: *N[C@H](C)Cc1cnc[nH]1] | OH | H | 1 | |
| 261 | H | F | H | —CONHMe | 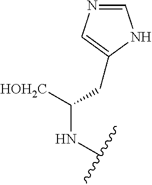 [SMILES: *N[C@H](CO)Cc1cnc[nH]1] | H | OH | 1 | |
| 262 | H | F | H | —CONHMe | 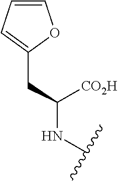 [SMILES: *N[C@@H](Cc1ccco1)C(=O)O] | H | OH | 1 | |
| 263 | H | H | H | —CONHMe | 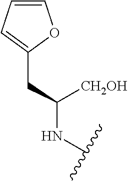 [SMILES: *N[C@H](CO)Cc1ccco1] | OH | H | 1 | |
| 264 | H | H | H | —CO₂Et | 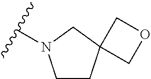 [SMILES: *N1CCC2(COC2)C1] | OH | H | 1 | |
| 265 | H | H | H | —CONHMe | 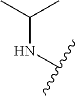 [SMILES: *NC(C)C] | OH | H | 1 | |
| 266 | H | H | H | —CO₂Et | 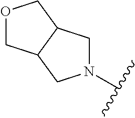 [SMILES: *N1CC2COCC2C1] | OH | H | 1 | |
| 267 | H | H | H | —CO₂Et | 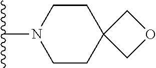 [SMILES: *N1CCC2(CC1)COC2] | OH | H | 1 | |
| 268 | H | H | H | —CONHMe | 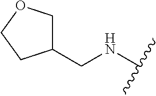 [SMILES: *NCC1CCOC1] | OH | H | 1 | |
| 269 | H | H | H | —CO₂Et | 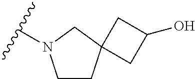 [SMILES: *N1CCC2(CC(O)C2)C1] | OH | H | 1 | |
| 270 | H | H | H | —CO₂Et | 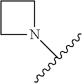 [SMILES: *N1CCC1] | N | OH | H | 1 |
| 271 | H | H | H | —CONHMe | 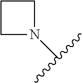 [SMILES: *N1CCC1] | OH | H | 2 | |
| 272 | H | H | H | —CO₂Et | 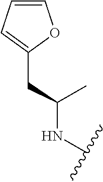 [SMILES: *N[C@H](C)Cc1ccco1] | OH | H | 1 | |
| 273 | H | H | H | —CO₂Et | 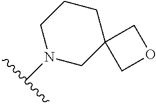 [SMILES: *N1CCCC2(COC2)C1] | OH | H | 1 | |
| 274 | H | H | H | —CONHMe | 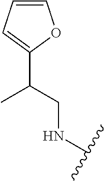 [SMILES: *NCC(C)c1ccco1] | OH | H | 1 | |
| 275 | H | H | H | —CONHMe | 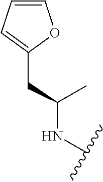 [SMILES: *N[C@H](C)Cc1ccco1] | OH | H | 1 | |

TABLE 7
Structure IV and Pks13 Inhibition Pharmacokinetic Data

| ID | IC₅₀ (μM) | MIC (μM) | ID | IC₅₀ (μM) | MIC (μM) | ID | IC₅₀ (μM) | MIC (μM) |
|---|---|---|---|---|---|---|---|---|
| 136 | >20 | 4 | 149 | 10 | 2.3 | 160 | 10 | NT |
| 137 | NA | NA | 150 | >40 | NT | 161 | 0.8 | 1.0 |
| 138 | >40 | NT | 151 | 10 | >20 | 162 | 2.7 | 2.5 |
| 139 | >40 | NT | 152 | >40 | NT | 163 | >40 | NT |
| 140 | >40 | NT | 153 | >40 | NT | 164 | NA | NA |
| 141 | 1.4 | 10.0 | 154 | >20 | NT | 165 | 0.8 | 0.08 |
| 142 | 40 | NT | 155 | >40 | NT | 166 | 0.5 | 6.1 |
| 143 | >20 | NT | 156 | >40 | NT | 167 | NA | NA |
| 144 | >40 | NT | 157 | >40 | NT | 168 | 20.0 | NT |
| 145 | 10.0 | NA | 158 | 20 | NT | 169 | >40 | NT |
| 146 | NA | NA | 159 | 20 | NT | 194 | >40 | NT |
| 147 | 3.5 | NA | 182 | NA | NA | 195 | | >40 |
| 148 | NA | NA | 183 | NA | NA | 196 | >20 | NT |
| 170 | 4.3 | NA | 184 | >10 | NT | 197 | 7.5 | NA |
| 171 | 0.5 | 2.7 | 185 | NA | NA | 198 | 5 | NA |
| 172 | 0.7 | 2.9 | 186 | 0.62 | 10 | 199 | 2.5 | 1.2 |
| 173 | 5.0 | NA | 187 | 0.5 | NA | 200 | 4.4 | 0.6 |
| 174 | 1.25 | 4.7 | 188 | NA | NA | 201 | 2.1 | <0.08 |
| 175 | 0.95 | 6.1 | 189 | 10.0 | NT | 202 | 1.6 | 15 |
| 176 | >40 | NT | 190 | >40 | NT | 203 | NA | NA |
| 177 | 1.0 | NA | 191 | 20 | NT | 204 | NA | NA |
| 178 | 2.0 | NA | 192 | >40 | NT | 205 | NA | NA |
| 179 | 6.5 | NA | 193 | 5.0 | NA | 206 | NA | NA |
| 180 | 1.9 | NA | 220 | NA | NA | 225 | >40 | NT |
| 181 | 2.5 | NA | 221 | NA | NA | 226 | >40 | NT |
| 207 | 10 | NT | 222 | NA | NA | 227 | >20 | NT |
| 208 | 4.0 | NA | 223 | NA | NA | 228 | 3.6 | 5.0 |
| 209 | 1.8 | 0.45 | 224 | NA | NA | 229 | 5.8 | 3.2 |
| 210 | 12 | NT | 73 | 2.1 | 2.5 | 230 | 40 | NT |
| 211 | NA | 0.33 | 74 | 20 | NT | 231 | 5.0 | 8.0 |
| 212 | 1.94 | 1.1 | 75 | >20 | NT | 232 | >20 | NT |
| 213 | 2.3 | 0.97 | 76 | >20 | NT | 233 | >10 | NT |
| 214 | NA | <0.08 | 77 | 6 | 3.3 | 234 | 5.3 | >20 |
| 215 | NA | <0.08 | 78 | 13.0 | NT | 235 | 2.3 | >40 |
| 216 | NA | NA | 79 | >20 | NT | 236 | >10 | NT |
| 217 | NA | NA | 80 | >40 | NT | 237 | >20 | NT |
| 218 | NA | 2.8 | 81 | >40 | NT | 238 | 40 | NT |
| 219 | NA | NA | 252 | 1.6 | NA | 265 | 0.3-.0.6 | NT |
| 239 | 10 | NT | 253 | 0.8 | NA | 266 | 10.0 | NT |
| 240 | >40 | NT | 254 | 2.7 | NA | 267 | >40 | NT |
| 241 | >20 | NT | 255 | 2.1 | NA | 268 | 40 | NT |
| 242 | >20 | NT | 256 | 1.5 | NA | 269 | NA | NA |
| 243 | >40 | NT | 257 | 1.25 | NA | 270 | NA | NA |
| 244 | >40 | 20-40 | 258 | 2.5 | NA | 271 | 7.0 | NT |
| 245 | 2.7 | >40 | 259 | 1.0 | NA | 272 | NA | NA |
| 246 | 20 | NT | 260 | 4.0 | NA | 273 | NA | NA |
| 247 | >40 | NT | 261 | 5.0 | 5.0 | 274 | NA | NA |
| 248 | 20 | NT | 262 | >40 | NT | 275 | NA | NA |
| 249 | >40 | NT | 263 | 10 | NT | | | |
| 250 | 2.4 | NA | 264 | 2.5 | NT | | | |
| 251 | 1.6 | NA | | | | | | |

Benzofuran derivatives which contain a basic moiety, such as, but not limited to an amine or a pyridine or imidazole ring, may form salts with a variety of organic and inorganic acids. Suitable pharmaceutically acceptable (i.e., non-toxic, physiologically acceptable) base addition salts for the compounds of the present invention include inorganic acids and organic acids. Examples include acetate, adipate, alginates, ascorbates, aspartates, benzenesulfonate (besylate), benzoate, bicarbonate, bisulfate, borates, butyrates, carbonate, camphorsulfonate, citrate, digluconates, dodecylsulfates, ethanesulfonate, fumarate, gluconate, glutamate, glycerophosphates, hemi sulfates, heptanoates, hexanoates, hydrobromides, hydrochloride, hydroiodides, 2-hydroxyethanesulfonates, isethionate, lactate, maleate, malate, mandelate, methanesulfonate, 2-naphthalenesulfonates, nicotinates, mucate, nitrate, oxalates, pectinates, persulfates, 3-phenylpropionates, picrates, pivalates, propionates, pamoate, pantothenate, phosphate, salicylates, succinate, sulfate, sulfonates, tartrate, p-toluenesulfonate, and the like. Benzofuran derivatives which contain an acidic moiety, such as, but not limited to a carboxylic acid, may form salts with variety of organic and inorganic bases. Suitable pharmaceutically acceptable base addition salts for the compounds of the present invention include, but are not limited to, ammonium salts, metallic salts made from calcium, lithium, magnesium, potassium, sodium and zinc or organic salts made from lysine, N,N-dialkyl amino acid derivatives (e.g. N,N-dimethylglycine, piperidine-1-acetic acid and morpholine-4-acetic acid), N,N′-dibenzylethylenediamine, chloroprocaine, choline, diethanolamine, ethylenediamine, meglumine (N-methylglucamine), t-butylamine, dicyclohexylamine, hydrabamine, and procaine.

The benzofuran derivatives may exist in their tautomeric form (for example, as an amide or imino ether). All such tautomeric forms are contemplated herein as part of the present invention.

The compounds described herein may contain asymmetric centers and may thus give rise to enantiomers, diastereomers, and other stereoisomeric forms. Each chiral center may be defined, in terms of absolute stereochemistry, as (R)- or (S)-. The present invention is meant to include all such possible isomers, as well as, their racemic and optically pure forms. Optically active (R)- and (S)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

Compositions and inhibitors of the present disclosure may also include a pharmaceutically acceptable carrier, in particular a carrier suitable for the intended mode of administration, or salts, buffers, or preservatives. Certain of the compounds disclosed herein are poorly soluble in water. Accordingly, aqueous compositions of the present disclosure may include solubility enhancers. Compositions for oral use may include components to enhance intestinal absorption. The overall formulation of the compositions and inhibitors may be based on the intended mode of administration. For instance, the composition may be formulated as a pill or capsule for oral ingestion. In other examples, the composition may be encapsulated, such as in a liposome or nanoparticle.

Compositions of the present disclosure may contain a sufficient amount of one or more inhibitors to cause inhibition of a bacterium (such as, for example, Mtb) to occur when the composition is administered to the bacterium. The amount can vary depending on other components of the composition and their effects on drug availability in a patient, the amount of otherwise required to inhibit the bacterium, the intended mode of administration, the intended schedule for administration, any drug toxicity concerns, drug-drug interactions, such as interactions with other medications used by the patient, or the individual response of a patient. Many compositions may contain an amount well below levels at which toxicity to the patient becomes a concern.

The amount of inhibitor present in a composition may be measured in any of a number of ways. The amount may, for example, express concentration or total amount. Concentration may be for example, weight/weight, weight/volume, moles/weight, or moles/volume. Total amount may be total weight, total volume, or total moles. Typically, the amount may be expressed in a manner standard for the type of formulation or dosing regimen used.

### Methods of Bacterial Inhibition

The present disclosure also provides methods of inhibiting a bacterium with an inhibitor as disclosed. In certain embodiments in which a bacterium is inhibited by administration of an inhibitor, the dosage and administration may be adequate to allow this inhibition. In certain embodiments, it may consist of regular administration of an amount of the inhibitor, to maintain a certain level of the inhibitory compound or compounds in the patient, the patient's blood, and/or a tissue in the patient. However, dosage amounts and the administration schedule may be adjusted based on other components of the composition and their effects on drug availability in a patient, the intended mode of administration, the intended schedule for administration, any toxicity concerns, and the patient's response to the inhibitor.

Without limiting the compositions and methods of administration described herein, in certain embodiments, the inhibitor can exhibit its inhibitory effect on a bacterium by directly or indirectly inhibiting fatty acid (e.g., mycolic acid) biosynthesis. In certain embodiments, this inhibition is mediated by binding of the inhibitor to a portion of a Pks13 enzyme in the bacterium. In certain embodiments, the portion of the Pks13 enzyme in the bacterium is the C-terminal thioesterase domain.

In certain embodiments, the inhibitors disclosed herein can be used for inhibition of a bacterium expressing Pks13. In certain embodiments of the present disclosure, the bacterium expressing Pks13 is a member of the suborder Corynebacterineae. In certain embodiments of the present disclosure, the bacterium expressing Pks13 is a Mycobacterium. In certain embodiments of the present disclosure, the bacterium expressing Pks13 is Mycobacterium tuberculosis. In certain embodiments of the present disclosure, the bacterium expressing Pks13 is Mycobacterium leprae. In certain embodiments of the present disclosure, the bacterium expressing Pks13 is Corynebacterium diptheriae. The bacterium can be located in any region of a patient, such as the lung. The bacterium may be latent or active.

In certain embodiments, the inhibitors disclosed herein can be used for inhibition of a bacterium expressing a close structural analog of Pks13. For example, in certain embodiments, the inhibitors disclosed herein can inhibit a bacterium expressing a close structural analog of Pks13 which is essential for the viability of the bacterium and contains a domain that is highly homologous to the Pks13 thioesterase domain.

The present disclosure is not limited by the precise target of inhibition. Inhibitors of the present disclosure can be used to inhibit any bacterium susceptible to inhibition by the inhibitor, irrespective of the precise mechanism of inhibition.

A bacterium present in a patient may be inhibited by delivering the inhibitor to the patient. The mode of delivery may be selected based on a number of factors, including metabolism of the inhibitor, the mode of administration of other drugs to the patient, the location and type of bacterium to be inhibited, the health of the patient, ability or inability to use particular dosing forms or schedules with the patient, preferred dosing schedule, and ease of administration. In specific embodiments, the mode of administration may be enteral, such as orally or by introduction into a feeding tube. In other specific embodiments, the mode of administration may be parenteral, such as intravenously or by inhalation.

The dosage amounts and administration schedule of the inhibitor can vary depending on other components of the composition and their effects on drug availability in a patient, the severity of infection, the intended schedule for administration, any drug toxicity concerns, and the patient's response to the drug. In certain embodiments, the amount and frequency of delivery may be such that levels in the patient remain well below levels at which toxicity to the patient becomes a concern. However the amount and frequency may also be such that the levels of inhibitor in the bacterium temporarily reach or continuously remain at a level sufficient to cause inhibition of the bacterium.

In certain embodiments, the administration of the inhibitor is calibrated to reach a threshold concentration in the plasma or tissue of a patient. Such calibration can take into consideration experimentally derived bioavailability, such as the exemplary study data provided below, as well as the mass of the patient. In certain embodiments, the threshold concentration is a proportion of the minimum inhibitory concentration (MIC). Representative MIC data for representative inhibitors is provided above.

In certain embodiments, and based on one or more of the considerations discussed, the unit dosage of the inhibitor is between about 1 mg/kg body weight to about 500 mg/kg body weight. In certain embodiments, the unit dosage is between about 5 mg/kg to about 350 mg/kg. In certain embodiments, the unit dosage is between about 10 mg/kg and about 200 mg/kg body weight.

In certain embodiments, the inhibitor has an MIC value against Mycobacterium tuberculosis of about 0.1 μM to about 50 μM, or about 0.3 μM to about 20 μM, or about 0.35 μM to about 12.5 μM, or about 1 μM to about 10 μM, or about 1 μM to about 15 μM, or about 1 μM to about 25 μM. In certain embodiments, the inhibitor has an MIC against Mtb of less than about 5 μM, or less than about 3 μM, or less than about 1 μM.

The present disclosure further includes methods of identifying whether an inhibitor is able to inhibit a bacterium. Such methods include preparing or obtaining such a derivative, applying it to a bacterium, and identifying that the derivative inhibits the bacterium.

The present disclosure further includes methods and uses of the inhibitors for making and/or manufacturing drugs for inhibition of a bacterium, including, for example a bacterium expressing Pks13, an Mtb bacterium, and any other susceptible bacterium.

In certain embodiments of the disclosed subject matter, any of the inhibitors recited above are obtained by chemical synthesis.

In certain embodiments, an inhibitor is administered with one or more antibacterial, antibiotic and/or chemotherapeutic drugs. For example, the inhibitor can be administered with one or more antimycobacterial drugs, including one or more antitubercular drugs. In certain exemplary embodiments, an inhibitor is administered with one or more drugs selected from the group consisting of isoniazid, rifampicin, pyrazinamide, ethambutol, rifapentine, rifabutin, streptomycin, kanamycin, and amikacin, capreomycin, viomycin, ciprofloxacin, levofloxacin, moxifloxacin, ofloxacin, gatifloxacin, para-aminosalicylic acid, cycloserine, terizidone, ethionamide, prothionamide, thioacetazone, linezolid, clofazimine, amoxicillin, clavulanate, imipenem, cilastatin, and clarithromycin.

In certain embodiments, two or more inhibitors may be administered simultaneously or in sequence. The administration of two or more inhibitors may help prevent the development of resistance to one or more of the inhibitors.

In certain embodiments according to the disclosed subject matter, an inhibitor is simultaneously co-administered with one or more antibacterial or antibiotic drugs, such as one or more antitubercular drugs. In further embodiments, an inhibitor is administered prior to and concomitantly with administration of one or more antibacterial or antibiotic drugs, such as one or more antitubercular drugs. In still further embodiments, an inhibitor is administered prior to, concomitantly with, and after administration of one or more antibacterial or antibiotic drugs, such as one or more antitubercular drugs.

Any of the inhibitors may be administered in an amount and for a time sufficient to inhibit a Pks13 enzyme, and/or to improve the ability of one or more antibacterial or antibiotic drugs to kill a pathogen. Frequency of administration may be such that a selected minimum amount of inhibitor remains biologically available at all times during the course of administration.

In certain embodiments in accordance with the disclosed subject matter, the inhibitors are administered orally (i.e. by enteral administration). Such oral administration can be by solid pill, liquid capsule, drink, nutritional supplement, meal, or any other means of oral administration. In alternative embodiments, the inhibitors are administered by parenteral administration, including, without limitation, intramuscular or subcutaneous injection and intravenous infusion.

Patients may include mammals, such as humans, pets, such as dogs and cats, and livestock, such as cattle, sheep, horses, and pigs. Patients may also include birds, such as chickens, turkeys, ducks, geese, quail and other poultry. Patients may be infected with a bacterium that is susceptible to an inhibitor according to the present disclosure, such as a bacterium expressing Pks13.

In a specific embodiment in accordance with the disclosed subject matter, a pharmaceutical composition may be formed containing an inhibitor as described above. The composition may contain additional compositions to stabilize or preserve the benzofuran derivative or derivatives. The composition may further contain compositions to increase uptake, particularly enteral uptake, or bioavailability of the inhibitor. Furthermore, the composition may contain other therapeutically active agents to be co-administered with the inhibitor or inhibitors, such as an additional antibacterial/antibiotic/antitubercular drug.

### Benzofuran Derivatives for Pks13 Inhibition

Pks13 was previously identified as a viable target for tubercular inhibition. As early as 2004, Pks13 was shown to be essential to survival of mycobacterial species. Recently, and as described in Sacchettini et al., Identification of New Drug Targets and Resistance Mechanisms in Mycobacterium Tuberculosis, PLOS ONE 8:9 (September 2013), incorporated by reference herein in its entirety, a benzofuran-based compound (“the test compound”) identified from a whole-cell screen was putatively found to target polyketide synthase Pks13, with an MIC of 2.0 μM against Mtb isolate H37Rv. The structure of the test compound is shown below.

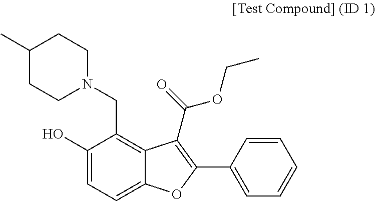 [SMILES: CCOC(=O)c1c(-c2ccccc2)oc2ccc(O)c(CN3CCC(C)CC3)c12]

The same study further identified 7 additional compounds exhibiting whole-cell Mtb inhibition, each having a distinct target of inhibition. An additional Mtb Pks13 inhibitor was recently described based on a thiophene core with an alternative putative binding site (fatty-acyl-AMP loading at the N-terminal of Pks13). Although the test compound was shown in a high-throughput screen to have whole-cell antitubercular activity, and sequencing of resistant mutants suggested Pks13 to be its putative intracellular target, the present disclosure establishes for the first time that Pks13 is indeed the target of the test compound and additional compounds in this series.

Thus the present disclosure establishes that Pks13, an essential polyketide synthase from Mtb, is the target of novel benzofuran-based inhibitors, and further provides a novel genus of Pks13 inhibitors. Compounds in this series are shown to specifically bind to and inhibit the TE domain of Pks13. The present disclosure further provides the first disclosure of the structural basis of inhibition of Pks13. The consensus structure of the inhibitors is also disclosed, and key structural sites for inhibitor activity are identified. On the basis of this structure-activity analysis, Inhibitors having potency at least an order of magnitude greater than the test benzofuran derivative were identified.

The inhibitors were found to be generally non-cytotoxic to mammalian cells and shown to be effective inhibitors of Mtb in vivo. Certain inhibitors, however, did exhibit toxicity in vitro and in vivo. These and other aspects of the disclosure are discussed in detail below in the Examples that follow.

### EXAMPLES

The following examples are provided to further illustrate certain embodiments of the disclosure. They are not intended to disclose or describe each and every aspect of the disclosure in complete detail and should be not be so interpreted. Unless otherwise specified, designations of cells lines and compositions are used consistently throughout these examples.

### Pks13 Thioesterase Domain Enzyme Inhibition by Benzofuran Derivative

To confirm inhibitor activity against the TE domain of Pks13, recombinant expression constructs were generated encompassing different start and stop sites for the TE domain of Mtb Pks13 based on the sequence alignments and the secondary structure from homologs. One of the constructs encompassing the residues 1451-1733 of Pks13 expressed well in E. coli as His₆-tagged protein. This construct contained three additional residues at its N-terminus that were derived from the TEV cleavage site on the vector. The protein was soluble even after removal of the tag and produced single diffraction quality crystals. An enzyme assay for thioesterase activity was developed using this purified recombinant TE domain (hereafter called Pks13-TE), with the fluorescent substrate 4-methylumbelliferyl heptanoate (4-MUH). Pks13-TE showed good esterase activity using 4-MUH as the substrate with a Kₘ˜20 μM, and k_{cat}/Kₘ˜7.2×10⁻⁴ μM⁻¹ min⁻¹.

The two mutant forms of the TE domain containing the resistance mutations from WGS (D1607N and D1644G) were also expressed and purified using an identical recombinant expression system as was used for the wild-type TE domain construct. As shown in Table 8, below, both mutants also exhibited thioesterase activity in the enzyme assay. Both mutations had an increased k_{cat}/Kₘ, with the D1607N mutant showing an increase of ˜1.7-fold and the D1644G mutant >3-fold over the wt-Pks13-TE.

TABLE 8
Kinetic Parameters of Wild-Type Pks13-TE and D1607N, D1644G
Pks13-TE Mutants

| | | | | Relative |
|---|---|---|---|---|
| Protein | Kₘ (μM)* | k_{cat} min⁻¹ | k_{cat}/Kₘ μM⁻¹ min⁻¹ | activity |
| Wt. | 19.5 ± 3.4 | 140 ± 9 × 10⁻⁴ | 7.2 ± 1.3 × 10⁻⁴ | 1 |
| D1607N | 8.5 ± 0.9 | 105 ± 3 × 10⁻⁴ | 12.3 ± 1.4 × 10⁻⁴ | 1.7 |
| D1644G | 6.9 ± 1.1 | 166 ± 6 × 10⁻⁴ | 24 ± 3.8 × 10⁻⁴ | 3.3 |

*Kinetics values were obtained by fitting the raw data to the Michaelis-Menten equation. All assays were carried out in triplicate. Results are presented as mean ± s.d.

The IC₅₀ of the test compound against Pks13-TE was determined to be 0.26 μM. The D1644G mutation increased the IC₅₀ of the test compound by more than 66-fold from 0.26 μM to 17.4 μM against the TE domain, and approximately 3-fold (to ˜0.76 μM) for the D1607N mutant.

Crystal Structure of Apo Pks13-TE and Pks13-TE in Complex with Test Compound

To determine the molecular basis of the inhibition of Pks13-TE observed with the test compound, the crystal structure of Pks13-TE in complex with the test compound was solved. To first solve the apo-structure, thin, plate-like crystals of apo-Pks13-TE were obtained by vapor-diffusion method in a condition with ammonium sulfate as the precipitant at pH 8.5. The crystals diffracted to a maximum resolution of 1.7 Å, and they belonged to the space group P2₁2₁2 with two molecules in the asymmetric unit (designated A and B). The phase solution for the apo-Pks13-TE structure was determined by the molecular replacement method, using the crystal structure of E. coli EntF (PDB code 3TEJ) as the search model. The final apo-structure contained two molecules, A and B, with 278 and 272 residues built out of 283 residues, respectively, and was refined to a resolution of 1.72 Å (R_{work}=17% and R_{free}=20%) with good stereochemistry. The apo, test compound-bound, and D1607N mutant Pks13-TE crystal structure modeling data are shown in Table 9 below.

TABLE 9
PKS13-TE Structure Refinement Data

| | Apo Pks13-TE | Pks13-TE:test | Pks13-TE (D1607N) |
|---|---|---|---|
| Data collection | | | |
| Space group | P2₁2₁2 | P2₁2₁2 | P2₁2₁2 |
| Cell dimensions | | | |
| a, b, c (Å) | 88.5, 106.7, 57.7 | 89.2, 109.5, 57 | 88.7, 108.9, 58.1 |
| α, β, γ (°) | 90, 90, 90 | 90, 90, 90 | 90, 90, 90 |
| Resolution (Å) | 39.19-1.72 (1.75-1.72)* | 39.5-1.94 (1.97-1.94) | 44.36-1.88 (1.91-1.88) |
| R_{merge} | 0.089 (0.55) | 0.172 | 0.141 |
| I/σI | 23.81 (4.33) | 18.8 (2.3) | 8.64 (0.53) |
| Completeness (%) | 99.8 (96.1) | 94.1 (93.29) | 92.5 (59.1) |
| Redundancy | 7.7 (6.7) | 6.6 (5.2) | 4.9 (2.6) |
| Refinement | | | |
| Resolution (Å) | 1.72 | 1.94 | 1.88 |
| No. reflections | 58442 | 39903 | 42945 |
| R_{work}/R_{free} | 0.172/0.201 | 0.191/0.235 | 0.182/0.222 |
| No. atoms | | | |
| Protein | 4278 | 4218 | 4258 |
| Ligand/ion | 27 | 63 | 44 |
| Water | 387 | 206 | 293 |
| B-factors | | | |
| Protein | 22 | 47 | 35 |
| Ligand/ion | 31 | 48 | 54 |
| Water | 28 | 45 | 40 |
| R.m.s. deviations | | | |
| Bond lengths (Å) | 0.011 | 0.007 | 0.014 |
| Bond angles (°) | 1.35 | 1.19 | 1.44 |

*Values in parentheses are for highest-resolution shell. Data from 1 crystal was used to solve each structure.

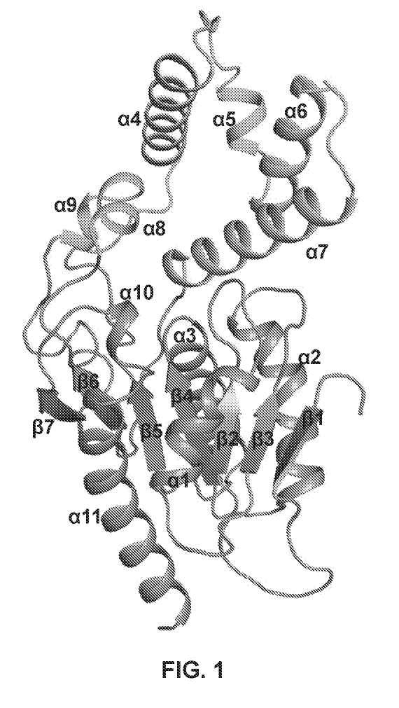

As shown in FIG. 1, the Pks13-TE structure is divided into two domains (lid and core), with the larger core domain (residues 1451-1570, 1646-1660 and 1680-1733) possessing a canonical α/β-hydrolase fold comprised of a central seven-stranded β-sheet (β1-β7) flanked by four α-helices (α1-α3 and all) with the N-terminal β1 strand anti-parallel to other strands. The smaller lid domain (residues 1575-1645) is inserted between strands β5 and β6 of the core domain and consists of four α helices, α4-α7, adjacent to the core domain. Two small helices, α8 and α9, (residues 1665-1675) that are present on a long loop between strands β6 and β7 of the core domain also form part of the lid domain. The topology of the TE domain in the Pks13-TE-test compound complex structure is very similar to the apo-enzyme structure (RMSD of 0.94 Å over 272 paired Cₐ atoms).

Analysis of the TE1451 structure with VAST server uncovered >100 structures belonging to thioesterase, hydrolase and lipase class enzymes with structural alignment scores well above the threshold of significance (VAST −log(p)>10). The top hit (VAST −log(p)>15) in the structural alignment was the thioesterase of fengycin synthesis, a non-ribosomal peptide synthetase (PDB code 2CB9) from B. subtilis. However, regardless of the enzyme class, the structural similarity was within the α/β-hydrolase core domain; the lid domain did not show significant structural conservation among the hits. In a VAST search using only the coordinates of the lid domain (1575-1645), only three hits were returned with alignment scores well below the significance threshold (VAST −log(p)<4) which had no structural or functional relationship with the lid domain. This finding is consistent with the role of lid domains in substrate recognition and binding in a variety of α/β-hydrolase enzymes, which accounts for their highly variable substrate specificities.

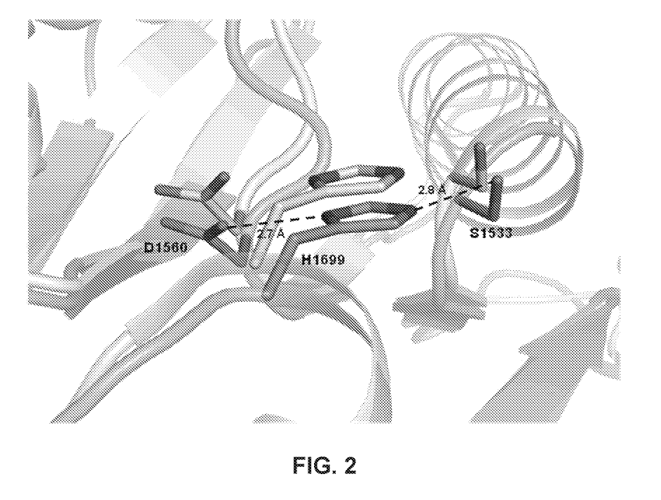

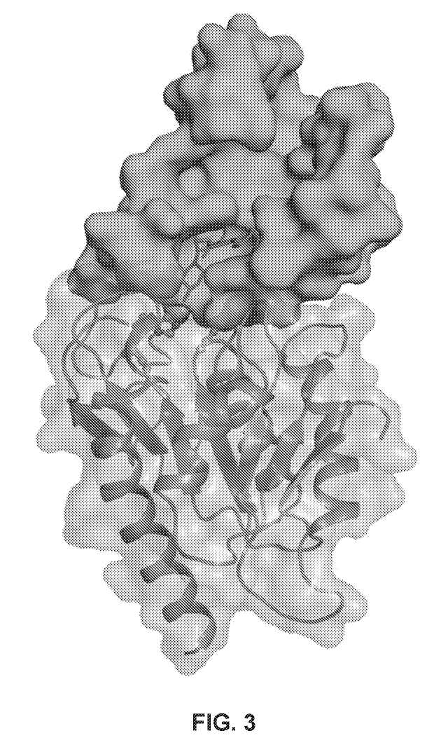

The active site of Pks13-TE was identified as a canonical Ser-His-Asp catalytic triad, similar to other α/β-hydrolase thioesterases. By superimposition of the Pks13-TE structure on the E. coli EntF structure (PDB code 3TEJ) as shown in FIG. 2, Ser1533 was identified as the active site nucleophile, and Asp1560 and His1699 as the other two members of the catalytic triad. The active site pocket is formed at the interface between the two domains, and it is situated at the proximal end of a long surface groove in the lid domain. This groove spans the full length of the lid domain (˜30 Å) with a total surface area ˜1290 Å², as calculated using the CASTp server. The active site of the Pks13-TE domain is shown in FIG. 3. The residues lining this groove are primarily hydrophobic, suggesting it could bind long-chain fatty acid substrates. A similar hydrophobic surface groove (˜20 Å long) leading to the active site serine was also observed in the α-helical lid domain of bovine palmitoyl-protein thioesterase 1 bound to palmitate. Additionally, a fragment of polypropylene glycol (C₁₂O₅H₂₅), an additive in the crystallization buffer, was observed bound in the active site of the apo-TE domain structure. Thus, the structure strongly suggests that the surface groove presents the substrate-binding site that can accommodate the meromycolate product and position it for de-esterification by the catalytic triad.

Next, crystals of Pks13-TE complexed to the test compound were obtained by soaking the apo-Pks13-TE crystals with the test compound. The complex structure was determined by molecular replacement to a resolution of 1.94 Å (space group P2₁2₁2 and unit cell similar to apo-Pks13-TE) with 273 and 274 residues built out of a total of 283 residues for molecules A and B, respectively, and refined to a final R_{work}=19% and R_{free}=23%. The test compound-bound Pks13-TE crystal structure data are also provided in Table 9 above.

As shown in FIG. 3, the test compound binds at the mouth of the substrate-binding groove, approximately 6 Å from the catalytic site. Binding of the inhibitor thus effectively blocks access of the substrate to the active site. The four different substituents attached to the benzofuran scaffold interact mainly with the residues from helix α7 of the lid domain (Gln1633, Ser1636, Tyr1637, Asn1640, Arg1641, Ile1643 and Asp1644) and the two supporting helices α8-α9 along with the loop that connects them to strand (36 of the core domain (Tyr1663, Ala1667, Phe1670, Glu1671 and Tyr1674). These binding interactions are summarized in Table 10 below

TABLE 10
Interactions of Test Compound with Pks13-TE Domain Residues

| TE domain residue | Type of interaction | Interacting group |
|---|---|---|
| Phe1670 | Planar stacking, | Benzofuran core |
| | hydrophobic | |
| Asp1644 | Hydrogen bond (2.4 Å) | —OH at C-5 |
| Asn1640 | Hydrogen bond (3 Å) | Basic N of piperidine |
| | | at C-4 |
| Gln1633, Ser1636 | van der Waals | Phenyl at C-2 |
| Tyr 1674, Tyr1663, | van der Waals | Piperidine ring at C-4 |
| Ala1667 | | |
| Glu1671 | van der Waals | —OH at C-5 |
| Tyr1582, Tyr1637 | van der Waals | Phenyl ring of core |
| Arg1641, Ile1643 | Hydrophobic | Phenyl ring of core |
| | | and methyl of piperidine |

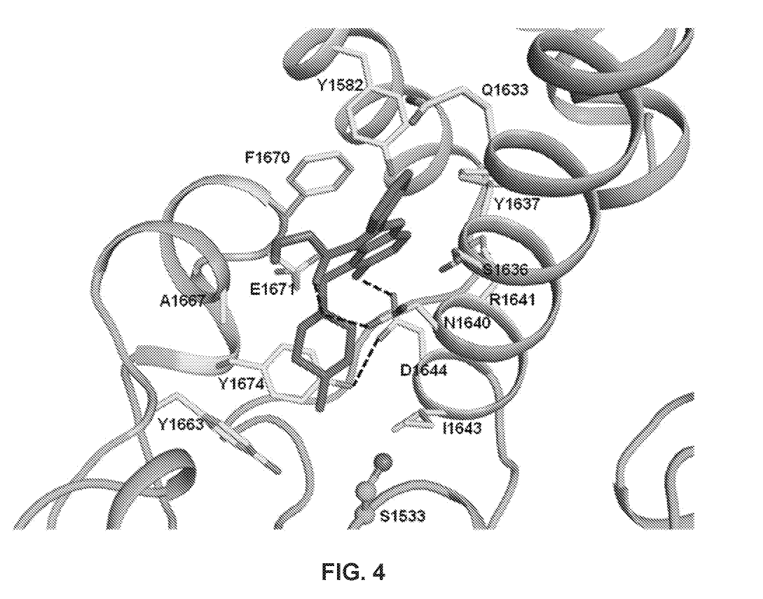

Several changes occur upon binding of the test compound, as depicted in FIG. 4. Most notably, Phe1670, located at the end of helix α8, flips to form a planar stacking interaction (3.6-3.7 Å) with the furan ring of the benzofuran and hydrophobic interactions with the phenyl component of the fused-ring system. The Phe1670 side chain also forms van der Waals interactions with the phenyl ring and the ethyl group of the ethyl ester attached to the test compound, respectively (˜4 Å).

The side chains of Tyr1582 and Tyr1637 also participate in van der Waals interactions with the phenyl ring of the benzofuran core (3.7-3.8 Å). One of the key interactions for binding of the test compound involves Asp1644, which is located at the end of helix α7 of the lid domain. The carbonyl oxygen of Asp1644 forms a strong hydrogen bond (2.4 Å) with the hydroxyl of the benzofuran compound. In addition to the direct interactions with the benzofuran, a carboxylate oxygen at Asp1644 also forms a hydrogen bond with the hydroxyl of Tyr1674 (2.7 Å), helping to orient it to form a face-on van der Waals interaction with the piperidine ring. The basic nitrogen of the piperidine ring acts as a bifurcated donor, making one hydrogen bond with the side chain oxygen of Asn1640 (3 Å) and another with the carbonyl oxygen of the ethyl ester (3.3 Å). Other residues that participate in the van der Waals interactions with the test compound are Gln1633 and Ser1636, with the phenyl ring; Tyr1663 and Ala1667, with the piperidine; and Glu1671, with the hydroxyl. Arg1641 and Ile1643 form hydrophobic interactions with the phenyl ring of the benzofuran core and the methyl group of the piperidine, respectively.

Thus the benzofuran binds non-covalently in a hydrophobic cleft between the core α/β-hydrolase and helical-lid domains, at the entrance of the catalytic chamber of the TE domain. This establishes the mechanism of inhibition, as it effectively blocks access of the meromycolate substrate to the active site Ser1533. In contrast, inhibitors of the human FAS TE domain were previously reported to exert their inhibitory effects by forming a covalent adduct that mimics the acyl-enzyme intermediate.

Structure Activity Relationship and Potency Enhancement of Benzofuran Derivatives

To establish that compounds similar to the test compound also exhibit inhibition, and to develop a structure-activity relationship (SAR) for this series, second-generation structural analogs of the test compound were evaluated for inhibition of Pks13-TE activity in vitro. The analogs were selected and evaluated to test the effects of chemical modifications at each of the four substituent positions of the benzofuran scaffold as shown below.

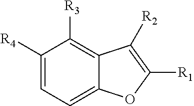

These compounds are shown in Table 11 below with their observed pharmacokinetics against Pks13-TE

TABLE 11
Second-Generation Inhibitors for Structure-Activity Relationship Analysis

| ID | R₁ | R₂ | R₃ | R₄ | IC₅₀ (μM)* | MIC (μM)* |
|---|---|---|---|---|---|---|
| 1 | 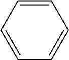 | 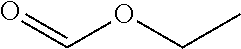 | 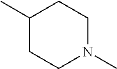 | OH | 0.26 ± 0.03 | 2.3 ± 0.2 |
| 2 | 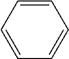 | 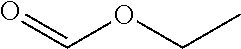 | 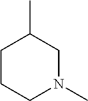 | OH | 0.12 ± 0.02 | 4.4 ± 0.2 |
| 3 | 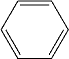 | 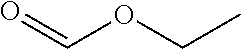 | 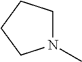 | OH | 0.24 ± 0.02 | 4.1 ± 0.1 |
| 4 | 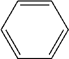 | 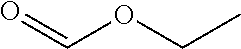 | 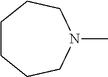 | OH | 0.28 ± 0.03 | 4.6 ± 0.03 |
| 5 | 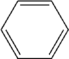 | 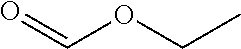 | 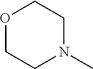 | OH | 0.71 ± 0.05 | 13.3 ± 1.6 |
| 109 | 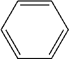 | 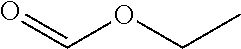 | 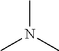 | OH | 1.57 ± 0.15 | 7.3 ± 0.3 |
| 6 | 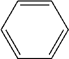 | 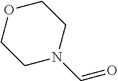 | H | OH | No binding | ND |
| 7 | 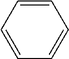 | 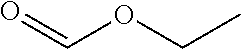 | 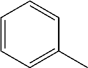 | OH | 11.9 ± 2.3 | >40 |
| 13 | 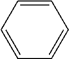 | 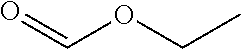 | 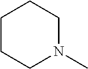 | OH | 0.26 ± 0.04 | 0.4 |
| 14 | 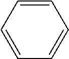 | 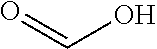 | 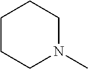 | OH | 6.6 ± 0.7 | NI |
| 16 | 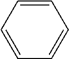 | 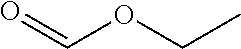 | 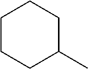 | OH | 19.6 ± 1.4 | 5.2 |
| 17 | 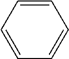 | 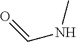 | 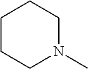 | OH | 0.29 ± 0.01 | 0.2 |
| 22 | 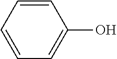 | 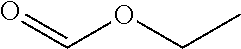 | 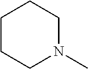 | OH | 0.17 ± 0.02 | 1.2 |
| 23 | 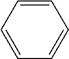 | 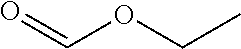 | 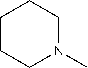 | MeO | 35.8 ± 2.2 | ND |
| 24 | 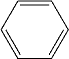 | 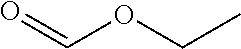 | 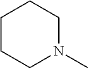 | H | 2.0 ± 0.1 | 16 |

*Values are shown as mean ± s.d. of triplicates.
MeO: methoxy; ND: not deteremined; NI: no inhibition.

IC₅₀ values were determined using the Mtb Pks13-TE domain as described in the methods section below. MIC values were determined for mc²7000 Mtb isolates in liquid medium in 96-well plates.

The SAR studies for the R₃ position were designed to investigate the effect of ring planarity and the role of the N-atom in inhibitor potency. On the basis of the Pks13-TE-test compound complex structure, it was hypothesized that the puckered piperidine might be replaceable by a phenyl ring, which could π-stack with Tyr1674. However, neither compound H having a phenyl group nor compound K having a puckered cyclohexyl group showed significant enzyme inhibition activity, exhibiting a loss of activity by >40- and >70-fold respectively, when compared to the test benzofuran compound. It was found that other saturated heterocycles (compounds B-E) maintained activity. The IC₅₀ values ranged from 0.12-1.57 μM against the TE domain). IC₅₀ data shows that compound B was more potent (0.12 μM) compared to the test compound (0.26 μM) against Pks13-TE. The only difference between these two positional isomeric compounds is that the methyl group on the piperidine ring is in para position in the test compound and in meta position in B. When the piperidine ring was replaced in the des-methyl analog I, it showed an IC₅₀ (0.3 μM) similar to the test compound. Compounds C and D have five- and seven-membered rings, respectively, at R₃, and exhibited IC₅₀ values similar to the test compound, whereas compound E, which has a six-membered morpholine ring with a polar oxygen atom, has ˜3-fold higher IC₅₀ against Pks13-TE compared to the test compound. In contrast to the compounds with cyclic amine substituents at R₃, compound F (having acyclic dimethyl amine at R₃) exhibited >6-fold loss in inhibitory activity relative to the test compound. In comparison, compound G, which lacks a substitution at R₃ altogether and has a morpholine ring at R₂ in place of the ethyl ester, did not show any appreciable binding to Pks13-TE, even at a concentration of 30 μM.

The structural basis for the inhibition of Pks13-TE by analogs of the test compound with replacement heterocycles at R₃ was determined by solving crystal structures of inhibitor complexes. The structures of Pks13-TE-analog binary complexes were refined using the Pks13-TE-apo structure. There was a clear positive |F₀|-|F_{c}| difference electron density for inhibitors C-F, which were in a very similar position in the substrate-binding groove. Ligands were fit into the electron density, and the structures were built and refined to a resolution of 2 Å with good stereochemistry. These structures are shown in FIG. 4. The protein components of all the complexes exhibit a nearly identical overall structure as compared to the Pks13-TE-test compound complex (RMSD of 0.4 Å for compound D, 0.7 Å for compound C, and 0.9 Å for compounds E and F vs. test compound complex over 264 paired C_{α} atoms). In the structures, the variation in the tertiary amine group at R₃ of the second-generation analogs did not affect their mode of binding. Crystal structure data and refinement statistics for inhibitors C-F in complex with Pks13-TE are provided in Table 12 below.

TABLE 12
Pks13-TE - Inhibitor Crystal Structure Data and Refinement Statistics

| | Pks13-TE:3 | Pks13-TE:4 | Pks13-TE:5 | Pks13-TE:6 |
|---|---|---|---|---|
| Data collection | | | | |
| Space group | P2₁2₁2 | P2₁2₁2 | P2₁2₁2 | P2₁2₁2 |
| Cell dimensions | | | | |
| a, b, c (Å) | 89.4, 109.4, 56.9 | 89, 110.2, 57.5 | 88.9, 109.7, 57.4 | 88, 109.4, 57 |
| α, β, γ (°) | 90, 90, 90 | 90, 90, 90 | 90, 90, 90 | 90, 90, 90 |
| Resolution (Å) | 33.76-1.99 (2.02-1.99)* | 48.33-2.04 (2.12-2.04) | 48.2-2.05 (2.10-2.05) | 46.45-1.99 (2.04-1.99) |
| R_{merge} | 0.114 | 0.158 | 0.158 | 0.124 |
| I/σI | 14.1 (1.5) | 9.0 (0.8) | 11.5 (1.6) | 9.9 (1.3) |
| Completeness (%) | 87.8 (84.4) | 87.6 (69.7) | 94.0 (91.6) | 99.0 (91.6) |
| Redundancy | 5.2 (4.5) | 4.3 (3.7) | 5.5 (4.6) | 4.3 (4.1) |
| Refinement | | | | |
| Resolution (Å) | 1.99 | 2.04 | 2.05 | 1.99 |
| No. reflections | 34374 | 32129 | 33649 | 38083 |
| R_{work}/R_{free} | 0.196/0.24 | 0.208/0.238 | 0.201/0.237 | 0.187/0.215 |
| No. atoms | | | | |
| Protein | 4220 | 4239 | 4204 | 4236 |
| Ligand/ion | 54 | 34 | 28 | 50 |
| Water | 152 | 99 | 208 | 386 |
| B-factors | | | | |
| Protein | 44 | 56 | 34 | 34 |
| Ligand/ion | 33 | 79 | 33 | 29 |
| Water | 45 | 53 | 34 | 38 |
| R.m.s. deviations | | | | |
| Bond lengths (Å) | 0.009 | 0.005 | 0.007 | 0.008 |
| Bond angles (°) | 1.28 | 0.98 | 1.10 | 1.14 |

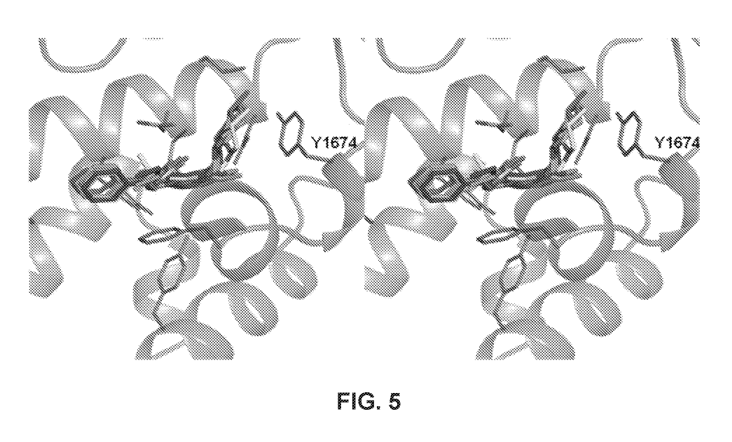

Binding of the test compound and compounds C-F to the Pks13-TE domain is illustrated in FIG. 5. The crystal structures revealed that the different cyclic amine groups at the R₃ position formed stacking interactions with Tyr1674 in a manner similar to the test compound. The small differences in the IC₅₀ values between the test compound and compounds C and D can be attributed to variations in the strength of the stacking interactions of the planar side chain of Tyr1674 with the variably puckered rings at R₃, similar to the sugar-ring Tyr interactions in the carbohydrate/sugar binding proteins. In the Pks13-TE-E complex structure, it was observed that the polar cyclic amine at R₃ also stacked with Tyr1674; however, it formed only van der Waals interactions with side chains of Ile1643 and Tyr1663 (4-4.3 Å). Contrary to expectations, the oxygen atom of the R₃ morpholine ring of compound E did not form a hydrogen bond interaction with the hydroxyl of Tyr1663, as its rotamer conformation positioned the hydroxyl at a distance of 4.2 Å. The effect of the reduced interactions of compound E on its inhibitory activity was reflected in a ˜3-fold increase in its IC₅₀ (0.71 μM) compared to the test compound (IC₅₀ 0.26 μM). In contrast to the cyclic groups at R₃, the presence of an acyclic dimethyl amine group at R₃ in compound F was expected to abolish the stabilizing stacking interaction with Tyr1674, and it was confirmed in the Pks13-TE-F complex structure. The structure also revealed that the smaller size of the side group at R₃ led to reduced van der Waals and hydrophobic interactions with Tyr1674, Tyr 1663 and Ile1643. As a consequence of this loss of interactions, there was >6-fold decrease in the inhibitory activity of F against Pks13-TE.

Since the ethyl ester at position R₂ can be hydrolyzed to the corresponding acid by broad-specificity esterases inside Mtb, it is plausible that the carboxylic acid form of test compound is the active compound. This hypothesis was tested by synthesizing compound J, the acid analog of compound I. Compound J had an IC₅₀ of 6.6 μM, a ˜20-fold loss in activity compared to the ester-containing analog compound I. The ethyl ester represents a pharmacological liability, however, as it could also be subject to hydrolysis by serum esterases, leading to the production of the less-active acid form of the inhibitor. Thus compound L, a methyl-amide analog of compound I, was synthesized. Compound L showed similar enzyme inhibitory activity (IC₅₀ 0.3 μM) as the ethyl ester-containing compound I and the test compound. Thus, the replacement of the ethyl ester at the R₂ position with an amide group maintained the inhibitor potency, while conferring to it the desired metabolic stability.

Testing of different substituents at the R₄ position demonstrated that the phenolic OH is important for inhibitor binding. This hydroxyl group forms a strong hydrogen bond with Asp1644, and hence is expected to contribute significantly to affinity. Two analogs of compound I were synthesized in which the R₄ OH was substituted with either a methoxy group (compound N) or a hydrogen atom (compound O). The IC₅₀ for compound N was determined to be ˜36 whereas it was 2 μM for compound O. Thus, introduction of a bulkier group and the removal of hydrogen bonding capability in compound N caused a >100-fold decrease in its inhibitory activity, while compound O showed ˜7-fold decrease in activity due to substitution of the R₄ OH with a hydrogen atom. It is possible that other hydrogen-bond donating groups at this or the adjacent position on the benzofuran ring could also yield inhibitors with high affinity.

The structure of the Pks13-TE complex with the test compound indicated that the side-chain amide of Gln1633 on helix α7 is positioned at a distance of ˜4 Å from the distal end of the R₁ phenyl ring. On the basis of this observation, compound M was synthesized containing an OH group at the C-4 of the R₁ phenyl ring, to determine if the modification resulted in additional hydrogen bond interactions. Compound M exhibited a slightly better IC₅₀ (0.19 μM) compared to the non-OH substituted compound I (IC₅₀: 0.3 μM). Thus, the hydroxylation of the R₁ phenyl ring at the para-position increases potency.

### Whole-Cell Activity of Benzofuran Derivative Inhibitors Against Mtb

Whole-cell assays show that the ester-analogs of the test compound, but not the corresponding acids, have Mtb growth-inhibition activity. The analogs were tested for anti-Mtb activity in a whole-cell growth inhibition assay using the Mtb strain mc²7000. All compounds that were active against Pks13-TE in vitro also inhibited the growth of Mtb bacteria to varying extents. Compounds B, C, and D had IC₅₀ values comparable to the test compound against Pks13-TE, but they exhibited ˜2-fold higher MICs against the bacteria. In comparison, compound E and F exhibited 6- and 3-fold higher MICs compared to the test compound. Notably, compound E had an IC₅₀ value >2-fold lower than compound F against Pks13-TE in vitro, but in whole cell testing its MIC was ˜2-fold higher than compound F. Since compound E is more polar, it is possible that this compound had reduced cell penetration, which subsequently resulted in an increase in its MIC. However, other effects like efflux or metabolism of compound E that can lead to an increase in its MIC cannot be ruled out. Among the synthesized analogs of the test compound, the whole-cell activity was completely abolished for the acid functionality containing analog J. When compound L was tested for the whole-cell activity, the MIC (0.2 μM) was found to be >10-fold lower than that of the test compound, suggesting that the replacement of the more labile ethyl-ester with the more stable amide group at the R₂ position significantly improved its whole-cell activity.

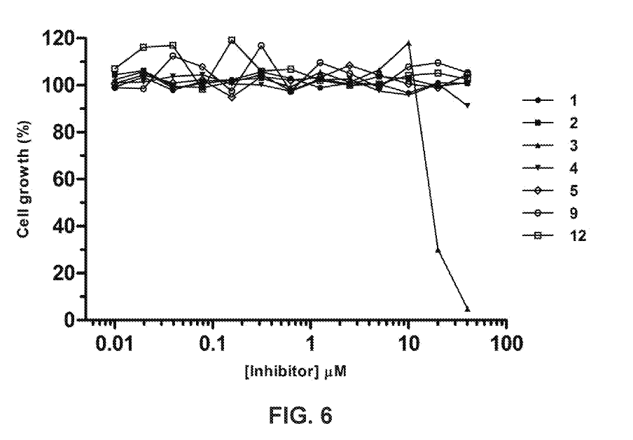

Experimental data indicated that compounds in this benzofuran series are not cytotoxic to human fibroblast cells. To assess the potential of the test compound and analogs that demonstrate good whole-cell activity (compounds B-E, I and L) as suitable candidates for further development, they were tested for cytotoxicity against human dermal fibroblast (HDF) cells using the resazurin dye assay. As shown in FIG. 6, the test compound and compounds B, D, E, I and L did not show any growth inhibition (relative to DMSO only control) in HDF cells up to 40 whereas compound C exhibited a half-maximum inhibitory concentration of >15 μM. The low cytotoxicity suggests that these compounds have good selectivity in vivo.

Structural Basis for Distinct Mechanisms of Resistance in D1607N and D1644G Mutants

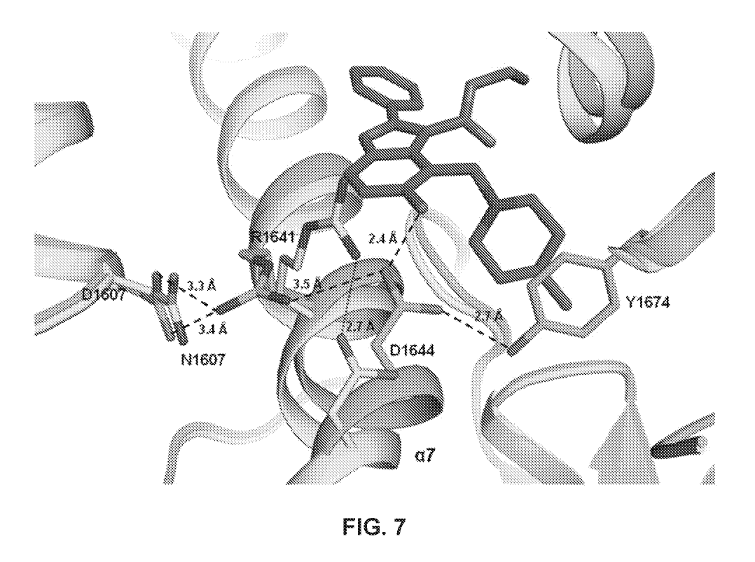

Crystal structures of the mutant proteins containing the resistance mutations identified by WGS suggest that they cause resistance to the test compound and its analogs through different mechanisms. The effect of D1644G mutation is direct, in that it removes the interaction with the R₄ hydroxyl of the test compound. The effect of the D1607N mutation is less apparent because it does not make any direct interactions with the test compound. Inspection of the interactions of Asp1607 in the D1607N mutant structure revealed that in the wtPks13-TE-test compound complex structure the side chain carbonyl oxygen atoms of Asp1607 form bidentate hydrogen bond interactions with the side chain amine group of Arg1641 (3.1 Å) which helps to anchor the C-terminal end of the α7 helix in a position that facilitates Asp1644 to form hydrogen bond interaction with the R₄ hydroxyl of the test compound and with Tyr1674. In contrast, and as shown in FIG. 7, the D1607N mutation disrupts this hydrogen bond interaction with Arg1641, allowing it to adopt a different rotamer conformation. This results in the loss of the anchoring effect by Arg1641 and causes the C-terminal end of helix α7 to move away from the substrate-binding groove. The resulting flexibility also causes Asp1644 to move away from the substrate-binding groove by 3 Å, altering its hydrogen bond interactions. The movement of Asp1644 disrupts its interaction with the test compound; however, it still is positioned within hydrogen bonding distance to Arg1641 (2.7 Å). Thus, the D1607N mutation introduces subtle changes in structural interactions, in contrast to the D1644G mutation, to disrupt TE domain interaction with the test compound and impart resistance to the bacterium.

In Vitro Mtb Cytotoxicity of Second- and Third-Generation Inhibitors

Based on the detailed SAR analysis discussed above, a third generation of representative benzofuran derivatives were synthesized. Pks13 enzyme inhibition of these inhibitors was tested as described herein. The structures and Pks13 enzyme inhibition data (MIC and IC₅₀) for representative inhibitors (test, second-, and third-generation) is provided in Tables 1-7 above.

Representative second- and third-generation inhibitors exhibiting potent Pks13 enzyme inhibition were further evaluated for whole cell inhibitory activity (cytotoxicity) against mc²7000 Mtb isolate cells. The structures of the representative inhibitors are provided with corresponding pharmacokinetic data in Table 13 below. The Non-Toxic Concentration data represent the highest concentrations of the test compounds exhibiting little or no cytotoxicity to human dermal fibroblast (HDF) cells, and the Survival vs. Control data represent percent survival of HDF cells relative to DMSO-only control. Where two values are provided, the first and second values in the Survival vs. Control column respectively correspond to percent HDF cell survival observed relative to DMSO-only control for the first and second values provided in the Non-Toxic Concentration column for the same compound.

TABLE 13
Representative high-potency inhibitor structure and pharmacokinetics

| | | | | | Survival |
|---|---|---|---|---|---|
| | | Pks13 | Pks13 | Non-Toxic | vs. |
| | | MIC | IC₅₀ | Concentration | Control |
| ID | Structure | (μM) | (μM) | (μM) | (%) |
| 8 | 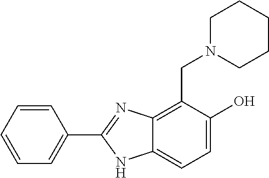 [SMILES: Oc1ccc2[nH]c(-c3ccccc3)nc2c1CN1CCCCC1] | 22 | 6.8 | 50, 25 | 78, 94 |
| 13 | 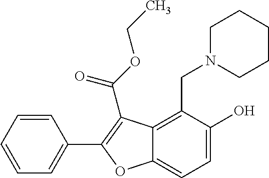 [SMILES: CCOC(=O)c1c(-c2ccccc2)oc2ccc(O)c(CN3CCCCC3)c12] | 0.4 | 0.26 | 50 | 105 |
| 17 | 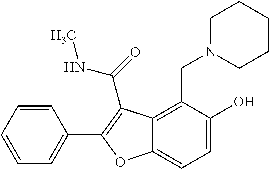 [SMILES: CNC(=O)c1c(-c2ccccc2)oc2ccc(O)c(CN3CCCCC3)c12] | 0.2 | 0.29 | 50 | 103 |
| 21 | 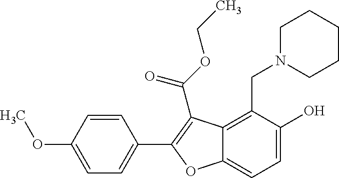 [SMILES: CCOC(=O)c1c(-c2ccc(OC)cc2)oc2ccc(O)c(CN3CCCCC3)c12] | 2.6 | 0.29 | 50 | 104 |
| 22 | 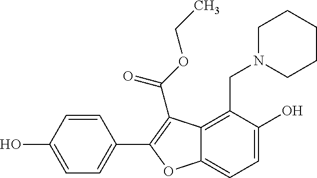 [SMILES: CCOC(=O)c1c(-c2ccc(O)cc2)oc2ccc(O)c(CN3CCCCC3)c12] | 0.6 | 0.19 | 50 | 103 |
| 27 | 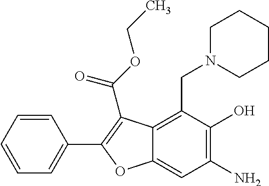 [SMILES: CCOC(=O)c1c(-c2ccccc2)oc2cc(N)c(O)c(CN3CCCCC3)c12] | 8 | 0.37 | 50, 25 | 77, 114 |
| 29 | 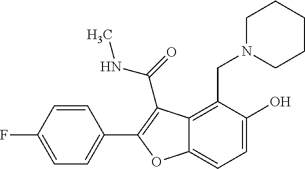 [SMILES: CNC(=O)c1c(-c2ccc(F)cc2)oc2ccc(O)c(CN3CCCCC3)c12] | 4.5 | 0.3 | 100, 50 | 89, 112 |
| 31 | 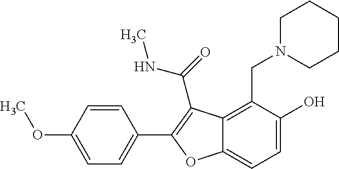 [SMILES: CNC(=O)c1c(-c2ccc(OC)cc2)oc2ccc(O)c(CN3CCCCC3)c12] | 1.1 | 0.26 | 50 | 111 |
| 32 | 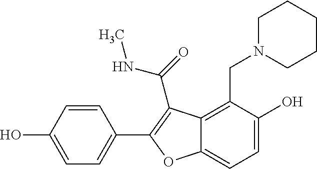 [SMILES: CNC(=O)c1c(-c2ccc(O)cc2)oc2ccc(O)c(CN3CCCCC3)c12] | 0.09 | 0.26 | 50 | 82 |
| 34 | 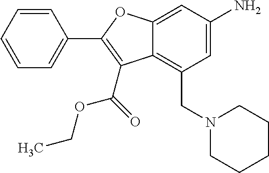 [SMILES: CCOC(=O)c1c(-c2ccccc2)oc2cc(N)cc(CN3CCCCC3)c12] | 9.2 | 4.5 | 100 | 82 |
| 38 | 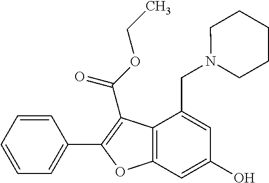 [SMILES: CCOC(=O)c1c(-c2ccccc2)oc2cc(O)cc(CN3CCCCC3)c12] | 6.7 | 0.45 | 100 | 111 |
| 39 | 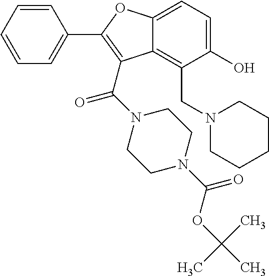 [SMILES: CC(C)(C)OC(=O)N1CCN(C(=O)c2c(-c3ccccc3)oc3ccc(O)c(CN4CCCCC4)c23)CC1] | 2.1 | 0.66 | 100 | 101 |
| 40 | 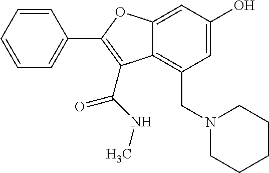 [SMILES: CNC(=O)c1c(-c2ccccc2)oc2cc(O)cc(CN3CCCCC3)c12] | 20 | 0.6 | 100 | 101 |
| 41 | 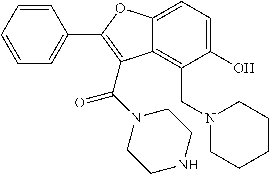 [SMILES: O=C(c1c(-c2ccccc2)oc2ccc(O)c(CN3CCCCC3)c12)N1CCNCC1] | 3.1 | 0.74 | 100 | 100 |
| 42 | 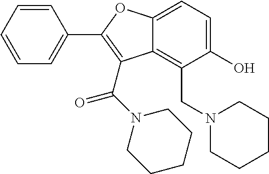 [SMILES: O=C(c1c(-c2ccccc2)oc2ccc(O)c(CN3CCCCC3)c12)N1CCCCC1] | >10 | 0.32 | 100 | 89 |
| 44 | 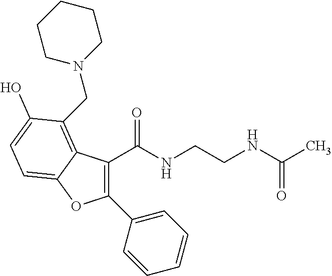 [SMILES: CC(=O)NCCNC(=O)c1c(-c2ccccc2)oc2ccc(O)c(CN3CCCCC3)c12] | >40 | 0.5 | 50 | 104 |
| 45 | 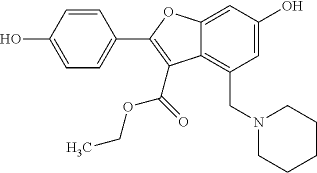 [SMILES: CCOC(=O)c1c(-c2ccc(O)cc2)oc2cc(O)cc(CN3CCCCC3)c12] | 1.1 | 0.44 | 100 | 89 |
| 47 | 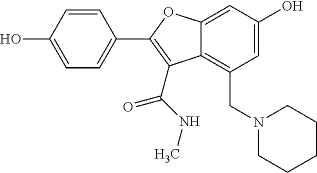 [SMILES: CNC(=O)c1c(-c2ccc(O)cc2)oc2cc(O)cc(CN3CCCCC3)c12] | 1 | 0.57 | 50 | 98 |
| 58 | 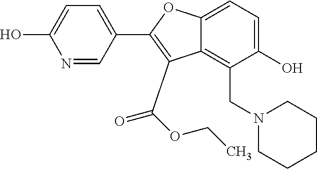 [SMILES: CCOC(=O)c1c(-c2ccc(O)nc2)oc2ccc(O)c(CN3CCCCC3)c12] | 4 | 0.36 | 50 | 95 |
| 61 | 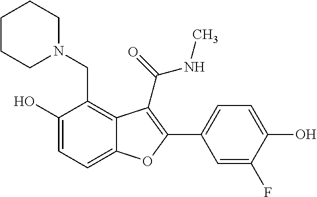 [SMILES: CNC(=O)c1c(-c2ccc(O)c(F)c2)oc2ccc(O)c(CN3CCCCC3)c12] | 0.5 | 0.33 | 50 | 94 |
| 62 | 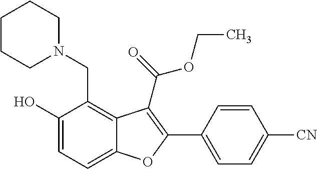 [SMILES: CCOC(=O)c1c(-c2ccc(C#N)cc2)oc2ccc(O)c(CN3CCCCC3)c12] | 1.5 | 0.27 | 6.25 | 90 |
| 63 | 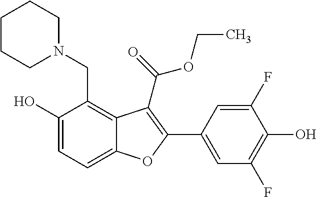 [SMILES: CCOC(=O)c1c(-c2cc(F)c(O)c(F)c2)oc2ccc(O)c(CN3CCCCC3)c12] | 7 | 0.5 | 25 | 99 |
| 64 | 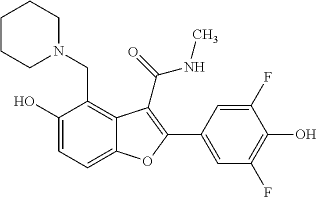 [SMILES: CNC(=O)c1c(-c2cc(F)c(O)c(F)c2)oc2ccc(O)c(CN3CCCCC3)c12] | NI (40 μM) | 1.5-1.9 | 100 | 94 |
| 65 | 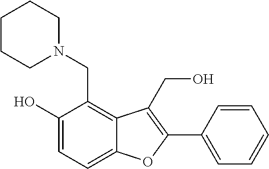 [SMILES: OCc1c(-c2ccccc2)oc2ccc(O)c(CN3CCCCC3)c12] | 6-12.5 | 0.24 | 25 | 95 |
| 67 | 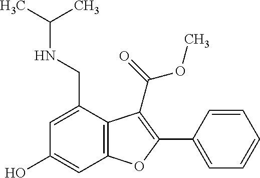 [SMILES: COC(=O)c1c(-c2ccccc2)oc2cc(O)cc(CNC(C)C)c12] | 9 | 0.7 | 25 | 97 |
| 69 | 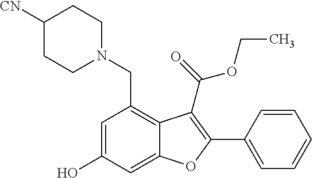 | 20 | 1.6 | 50 | 93 |
| 70 | 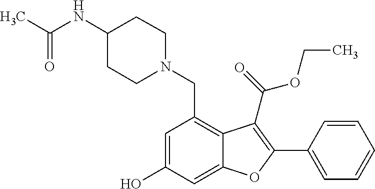 [SMILES: CCOC(=O)c1c(-c2ccccc2)oc2cc(O)cc(CN3CCC(NC(C)=O)CC3)c12] | >40 | 6.1 | 50 | 91 |
| 72 | 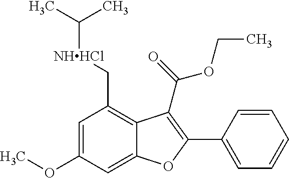 | 7.4 | >10 | 3.125 | 91 |
| 73 | 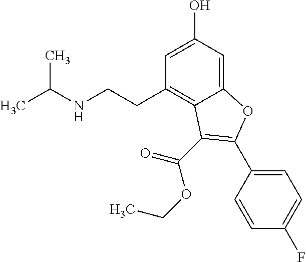 [SMILES: CCOC(=O)c1c(-c2ccc(F)cc2)oc2cc(O)cc(CCNC(C)C)c12] | 2.5 | 1.5-2.1 | 12.5 | 97 |
| 74 | 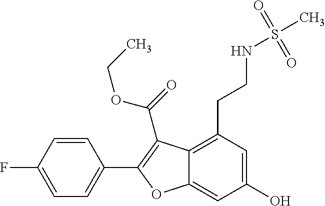 [SMILES: CCOC(=O)c1c(-c2ccc(F)cc2)oc2cc(O)cc(CCNS(C)(=O)=O)c12] | | 20 | 25 | 88 |
| 88 | 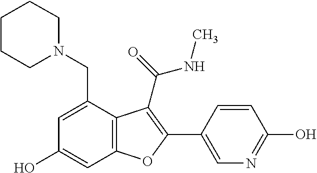 [SMILES: CNC(=O)c1c(-c2ccc(O)nc2)oc2cc(O)cc(CN3CCCCC3)c12] | | 2.8 | 50 | 96 |
| 102 | 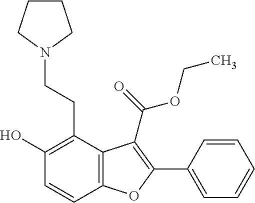 [SMILES: CCOC(=O)c1c(-c2ccccc2)oc2ccc(O)c(CCN3CCCC3)c12] | | 0.7 | 1.56 | 91 |
| 103 | 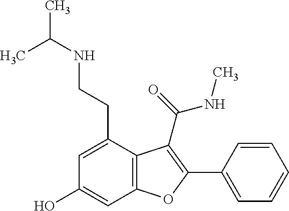 [SMILES: CNC(=O)c1c(-c2ccccc2)oc2cc(O)cc(CCNC(C)C)c12] | | 0.45 | 50 | 82 |

### Efficacy Against Drug Resistant Mtb Strains

The efficacy of Inhibitor 32 against drug resistant Mtb strains was tested. A non-drug resistant control strain, H37Rv, was tested as well. MDR strains are resistant to both isoniazid (INH) and rifampicin (RMP) and may also be resistant to other drugs. XRD strains were MDR strains that are also resistant to any fluoroquinolone, and to any of the three second-line injectables (amikacin, capreomycin, and kanamycin). Pre-XRD strains are MDR strains with additional resistance either a fluoroquinolone or an second-line injectable, but not both. Resistance is indicated in the drug profile of Table 14. The Mtb lineage and efficacy data are also presented.

TABLE 14
Efficacy Against Resistant Mtb

| | Drug | |
|---|---|---|
| Lineage | Profile | MIC (mM) |
| M. tuberculosis | Susceptible | 0.25 |
| Control | | |
| Atypical Beijing | XDR | 0.125 |
| Atypical Beijing | XDR | 0.125 |
| Typical Beijing | XDR | 0.25 |
| Typical Beijing | XDR | 0.25 |
| LAM | Pre-XDR | 0.125 |
| LAM | Pre-XDR | 0.25 |
| X1-Fam | Pre-XDR | 0.125 |
| X1-Fam | Pre-XDR | 0.125 |
| Beijing | MDR | |
| LAM 3 (F11) | RMP-mono | |
| S Fam (F28) | INH-mono | |

### In Vivo Safety and Efficacy of Benzofuran Derivative Inhibitors

Inhibitor 32 in Wild-Type Mice

To determine the in vivo safety and efficacy of representative inhibitor 32, the inhibitor was experimentally administered to Mtb-infected wild-type female Balb/C mice. The inhibitor was administered as a single dose of 300 mg/kg administered once daily five times per week for four weeks by oral gavage in 200 uL of canola oil. Efficacy was evaluated by determining log reduction in Mtb colony forming units (CFU) and relative light units (RLU) (an index of bacterial load detected by standard ATP-Luciferase assay) after 27 day Mtb incubation followed by 27 day treatment as described. Efficacy was evaluated with comparison to untreated and isoniazid-treated (25 mg/kg) mice.

Bacterial CFU and RLU data in the lung and spleen for the various treatment groups is provided in Table 15 below. The inhibitor showed activity comparable to that of the current front-line drug isoniazid in both the lungs and the spleen after 4 weeks of treatment. The log reduction in Mtb colony forming units in the lungs of Inhibitor 32 treated mice, relative to that of the untreated controls, was 0.88, which was statistically significant (p=0.01). Inhibitor 32 treatment reduced the spleen burdens by 2.23 logs relative to that of the untreated control (p<0.001). The isoniazid control treatment gave a 1.11 log reduction in the lungs and a 2.52 log reduction in the spleen, which is consistent with previous observations. These observations were consistent with histologic observations after sacrifice.

TABLE 15
Mtb CFU and RLU in Inhibitor 32 treated- and control mice

| | | Lung | | | | Spleen | | | |
|---|---|---|---|---|---|---|---|---|---|
| Treatment | n | CFU | SEM | RLU | SEM | CFU | SEM | RLU | SEM |
| Pre- | 5/5 | 7.38 | 0.07 | 4.84 | 0.08 | 5.52 | 0.09 | 2.75 | 0.11 |
| treatment | | | | | | | | | |
| Inhibitor | 5/5 | 5.21 | 0.12 | 2.7 | 0.09 | 2.64 | 0.17 | 1.69 | 0.08 |
| 32 | | | | | | | | | |
| Isoniazid | 5/5 | 4.98 | 0.07 | 2.37 | 0.04 | 2.35 | 0.08 | 1.59 | 0.06 |
| Untreated | 5/5 | 6.09 | 0.27 | 3.38 | 0.18 | 4.87 | 0.17 | 2.31 | 0.1 |

There was no statistically significant difference in CFU between isoniazid and Inhibitor 32 treated mice in either the lungs or the spleen, indicating that the two drugs performed equally well. The treated mice tolerated dosing well over the four-week course of treatment, and mouse weights of all groups remained stable over the course of treatment. Experimental data and statistical analyses are provided in Table 16 below.

TABLE 16
Statistical comparison of treatment efficacy

| | Diff of | | | | |
|---|---|---|---|---|---|
| Comparison | Means | p | q | P | P < 0.050 |
| Control vs. Isoniazid | 2.523 | 3 | 17.179 | <0.001 | Yes |
| Control vs. Inhibitor 32 | 2.228 | 3 | 15.17 | <0.001 | Yes |
| Inhibitor 32 vs. Isoniazid | 0.295 | 3 | 2.009 | 0.362 | No |

Inhibitor 32, Inhibitor 17, Inhibitor 31, and Inhibitor 47 in GKO Mice

Subsequently, the in vivo safety and efficacy of representative inhibitors, Inhibitor 32, Inhibitor 17, Inhibitor 31, and Inhibitor 47 was evaluated and compared in knockout mice engineered to lack expression of γ-interferon for rapid in vivo screening method for testing efficacy of inhibitors against acute infection of M tuberculosis. With the exception discussed below, efficacy was evaluated after 13 day Mtb incubation followed by once-daily administration for nine consecutive days. As before, each treatment was administered as a single drug by oral gavage of 200 μL in canola oil. Efficacy was evaluated by determining log reduction in Mtb colony forming units and relative light units (RLU) in comparison to untreated and isoniazid-treated (25 mg/kg) mice.

Significant differences in efficacy and drug tolerance were observed between the four inhibitors. Treatment with 300 mg/kg dosing of Inhibitor 32 resulted in a 1.18 log CFU reduction in the Lungs relative to that of the untreated controls which was statistically significant (p<0.001). In the Spleen, there was a 3.64 log CFU reduction compared to the untreated controls (p<0.001). Treatment with 100 mg/kg Inhibitor 31 resulted in a 0.84 log CFU reduction in the lungs vs. untreated controls (p<0.001), and a 1.54 log CFU reduction relative to the untreated controls (p<0.001) in the spleen. Inhibitor 47 (300 mg/kg dosing) did not show any activity in either the lungs or the spleen.

Inhibitor 17 treated mice, while initially dosed at 300 mg/kg, were dosed at 200 mg/kg beginning on day four of dosing due to apparent toxicity (one animal had a seizure and was euthanized). The remaining four animals were sacrificed on day 8 of dosing due to apparent toxicity which manifested on day 8 as seizures in two of the animals. One untreated control animal was also sacrificed on day 8 of dosing to serve as a comparator. The log CFU reduction versus the untreated control in the lungs was 0.53, and in the spleen 3.72. Because it was necessary to sacrifice these animals early, on day 8 of dosing, there was not the typical 24 hour period of drug clearance allowed prior to recovery and plating of the organ homogenates and it is therefore possible that drug carryover could give an erroneously low CFU. These data should therefore be interpreted with caution.

The isoniazid-treated controls showed a 3.02 log CFU reduction relative to the untreated control in the lungs, and a 4.16 log CFU reduction in the spleen, which is consistent with previous results.

Luciferase assay data correlated well with CFU data in both lungs and spleen, with the log RLUs approximately 2 logs lower than the CFU, which is consistent with that seen in previous GKO experiments. The lung RLU and CFU data in particular correlated closely. Inhibitor 32-treated mice showed a log reduction in RLU of 0.68, while isoniazid-treated mice showed a log reduction in RLU of 1.01. The spleen RLU data is less reliable because the CFU burden in both treatment groups was below the standard detection limits of the RLU luciferase assay (which is approximately 4.5-5 log CFU in this model). This would account for the lower correlation between the RLU and CFU data for the spleen.

Treatment with Inhibitor 31 resulted in a 0.84 log CFU reduction in the lungs vs. untreated controls (p<0.001), and a 1.54 log CFU reduction relative to the untreated controls (p<0.001) in the spleen. Luciferase assay data correlated well with CFU data in both lungs and spleen, with log RLUs approximately 2 logs lower than the CFU, which is consistent with that seen in previous GKO experiments.

Experimental data and statistical analyses are provided in Tables 18-18 below.

TABLE 17
In Vivo Efficacy Data for Representative Inhibitors

| Treatment | | Lung | | | | Spleen | | | |
|---|---|---|---|---|---|---|---|---|---|
| | n* | CFU | SEM | RLU | SEM | CFU | SEM | RLU | SEM |
| Pre- | 5/5 | 7.15 | 0.07 | 4.73 | 0.03 | 4.61 | 0.12 | 4.26 | 0.08 |
| treatment | | | | | | | | | |
| Untreated | 1/1 | 7.74 | N/A | 5.69 | 0.09 | 6.73 | N/A | 4.4 | 0.15 |
| Inhibitor | 4/4 | 7.21 | 0.09 | 4.24 | 0.08 | 3.01 | 0.11 | 1.85 | 0.06 |
| 17 | | | | | | | | | |
| Inhibitor | 5/5 | 6.99 | 0.14 | 3.88 | 0.1 | 3.01 | 0.16 | 1.81 | 0.08 |
| 32 | | | | | | | | | |
| Inhibitor | 5/5 | 7.33 | 0.12 | 5.06 | 0.07 | 5.11 | 0.22 | 2.59 | 0.15 |
| 31 | | | | | | | | | |
| Inhibitor | 5/5 | 8.39 | 0.13 | 6.09 | 0.12 | 6.84 | 0.19 | 4.45 | 0.13 |
| 47 | | | | | | | | | |
| Isoniazid | 5/5 | 5.15 | 0.03 | 3.51 | 0.11 | 2.49 | 0.1 | 1.77 | 0.05 |
| Control | 5/5 | 8.17 | 0.08 | 5.69 | 0.09 | 6.65 | 0.05 | 4.4 | 0.15 |

TABLE 18
Statistical Comparison of Inhibitor 31 and Inhibitor 32 Treatment Efficacy

| | | Diff of | | | |
|---|---|---|---|---|---|
| Comparison | Organ | Means | p | q | P |
| Control vs. Isoniazid (p = 4) | Lung | 3.013 | 4 | 28.988 | <0.001 |
| | Spleen | 3.644 | 4 | 24.83 | <0.001 |
| Control vs. Inhibitor 32 | Lung | 1.174 | 4 | 11.296 | <0.001 |
| (p = 4) | Spleen | 1.543 | 4 | 10.515 | <0.001 |
| Control vs. Inhibitor 31 | Lung | 0.834 | 4 | 8.025 | <0.001 |
| (p = 4) | Spleen | 2.623 | 4 | 17.874 | <0.001 |
| Inhibitor 31 vs. Isoniazid | Lung | 2.179 | 4 | 20.963 | <0.001 |
| (p = 4) | Spleen | 2.101 | 4 | 14.315 | <0.001 |
| Inhibitor 31 vs. Inhibitor 32 | Lung | 0.340 | 4 | 3.271 | 0.137 |
| (p = 4) | Spleen | 0.522 | 4 | 3.558 | 0.095 |

### Experimental Methods

Cloning and Overexpression of Mtb Pks13 TE Domain Construct

The TE domain constructs corresponding to the predicted TE domain in Mtb Pks13 gene (Rv3800c) were made by PCR from the Mtb H37Rv genomic DNA as the template. The amplified DNA fragments were incorporated into the pMCSG-19b vector by ligation independent cloning (LIC) to yield TEV protease cleavable N-terminal His₆-tagged TE domain constructs. The Pks13-TE-pMCSG-19b vectors were transformed into E. coli BL21(DE3)pLysS cells (Novagen) and the transformed cells were grown at 37° C. in LB media containing carbenicillin (100 μg/ml) and chloramphenicol (34 μg/ml) to an OD₆₀₀ of 0.6. Expression of TE constructs was induced with 0.5 mM IPTG, and cells were harvested after 16 hours of growth at 20° C.

The D1607N and D1644G mutants of Pks13 TE domain were constructed using the QuikChange site-directed mutagenesis kit (Stratagene). The mutations were confirmed by DNA sequencing. Mutant plasmids were transformed into E. coli BL21(DE3)pLysS cells, and mutant proteins were expressed by induction with 0.5 mM IPTG at 20° C. for 18 h.

Purification of Pks13 TE Domain

The harvested cells were resuspended in the lysis buffer (50 mM Tris-HCl pH 8.0, 0.5 M NaCl, 10% (v/v) glycerol, 1 mM β-mercaptoethanol (BME) and DNase) and lysed by French press. The resulting cell extract was clarified by centrifugation (15,000×g) for 1 hour at 4° C. The cleared supernatant was loaded onto a Ni-affinity column and the His-tagged TE domain constructs were eluted with a linear gradient of 10-250 mM imidazole in 20 mM Tris-HCl, pH 8.0 and 0.5 M NaCl. The peak fractions were pooled and the His-tag was cleaved by overnight incubation with TEV protease in dialysis buffer (20 mM Tris-HCl pH 8.0, 10% (v/v) glycerol and 1 mM DTT). The TEV cleaved protein was passed through Ni-column to remove any uncleaved His-tagged protein using 20 mM Tris-HCl (pH 8.0) with 100 mM NaCl and 1 mM BME. His-tag cleaved protein eluted in the flow-through and was concentrated for loading onto a Superdex-200 gel filtration column (GE Healthcare). The TE domain constructs eluted under a single peak as a monomer (˜32 kDa) from the gel filtration column and were >95% pure as observed by SDS-PAGE. The purified protein was concentrated to 20-25 mg/ml, flash-frozen and stored at −80° C. The TE domain mutants were purified using the same protocol as for the wild-type TE domain constructs. Both the mutants and the wild-type TE domain protein constructs have the amino acids SNA from the TEV cleavage site appended to the N-terminus.

Crystallization and Soaking with Ligands

Initial screening for crystallization conditions for the soluble TE domain constructs was done by sitting drop method using 1 μl of purified protein (15-20 mg/ml) and 1 μl of crystallization buffer from the well solution. After extensive screening, crystals were obtained for only the 283 residue long construct of the TE domain starting from residue 1451 in full length Pks13 (referred to as Pks13-TE in this paper). The Pks13-TE crystals were obtained in crystallization buffer containing 0.1 M Tris-HCl, pH 8.5 and 2.0-1.8 M ammonium sulfate as precipitant. The crystals were further optimized by using polypropylene glycol P-400 as an additive at 2%-5% (v/v) in the original condition. To obtain Pks13-TE-inhibitor complex crystals, soaking of the inhibitors was done by transferring apo-Pks13-TE crystals into a drop consisting of 0.1 M Tris-HCl, pH 8.5 and 2-2.2 M ammonium sulfate with 1-2.5 mM inhibitor added from a DMSO stock keeping the final DMSO concentration at <5%, and incubated at 18° C. and 4° C. for 4-20 hours.

Crystals of the TE11451:D1607N mutant were obtained by sitting drop method at 18° C. The crystallization drops contained an equal volume of the protein solution (15-20 mg/ml) and mother liquor (0.1 M HEPES, pH 7.5, 2%-4% (v/v) PEG 400, and 1.8-2 M ammonium sulfate), and the diffraction quality crystals were obtained within 2 weeks.

Data Collection and Processing

For diffraction data collection the crystals were cryo-protected using either Fomblin (Sigma) or 2.4 M malonate (Hampton Research) and flash frozen in liquid nitrogen. High resolution data was collected at a wavelength of 0.98 Å on the beamlines 19-ID and 23-ID at the Advanced Photon Source (APS) of the Argonne National Laboratory. All the data sets were processed and scaled with HKL2000. Analysis of the integrated and scaled data by Xprep indicated that Pks13-TE crystallized in P2₁2₁2 space group. Solvent content analysis indicated the presence of two molecules (V_{M} 2.16, V_{S} 43.2%) in the asymmetric unit.

Determination of Pks13-TE Atructures and Model Refinement

The structure of the TE domain was solved by molecular replacement method (MR) using E. coli EntF (PDB code 3TEJ), as search model. A single MR solution was obtained using Phenix AutoMR which was input into the AutoBuild wizard to generate the initial model for apo-Pks13-TE. The initial model was improved by further manual rebuilding in COOT. The final model was obtained after iterative cycles of model building and Phenix refinement with simulated annealing yielding a 1.72 Å resolution apo-Pks13-TE model with R_{cryst} of 17% and an R_{free} of 20% with good stereochemistry. The final refined apo-model has two chains, designated A and B, and 388 water molecules in the asymmetric unit.

To solve the Pks13-TE-inhibitor complex structures, as well as the D1607N mutant structure, only the protein atoms from chain A of the apo-Pks13-TE structure were used as search model in the initial rigid body refinement of the isomorphous P2₁2₁2 crystals in the Phenix Refine module. Inspection of electron density maps showed clear |Fₒ-F_{c}| positive difference density for the ligands which were fit into the density using Ligandfit routine in Phenix. The ligand model and geometry restraint files were created in ELBOW BUILDER of the Phenix suite. Iterative cycles of model building and NCS-restrained maximum likelihood refinement with simulated annealing in Phenix refine yielded high quality models for Pks13-TE-inhibitor complexes. Some of the residues in Pks13-TE structures at the flexible N- and C-termini, and the loops of the lid domain could not be built into the model due to the missing electron density, and some of the residues which showed ambiguous side chain electron density were modeled as alanines. In all of the structures >98% of residues are placed in the favored region of the Ramachandran plot as determined by MolProbity validation tool in Phenix.

Enzyme Assay

Activity of Pks13-TE was assessed using 4-methylumbelliferyl heptanoate (4-MUH, Sigma) as a fluorogenic substrate in a 96-well plate format. To make initial velocity measurements, Pks13-TE (1 μM) in 0.1 M Tris-HCl, pH 7 buffer was incubated with different concentrations of 4-MUH (2-150 μM in DMSO) in a 100 μl reaction volume, and the fluorescence of the hydrolyzed product 4-methylumbelliferone was read (excitation at 355 nm and emission at 460 nm) using PolarStar Omega plate reader (BMG Labtech) at 5-10 min intervals over 60-70 min. The reaction rate was observed to be linear in the measured range. 4-MUH in buffer alone was included as a control to quantify its background hydrolysis. Data points were plotted as an average of triplicates and each experiment was repeated 2-3 times independently. The initial velocity data was curve fit to Michaelis-Menten equation by nonlinear regression using Prism software (Graphpad) to determine the kinetic parameters Kₘ and Vₘₐₓ. The assay and data analysis for Pks13-TE mutants was done the same way as that for the wild-type protein with the 4-MUH concentration varying from 2 to 300 μM.

IC₅₀ Determination

To determine the potency of the test compound and its analogs against wt Pks13-TE, the compounds were tested at concentrations ranging from 0.012 to 20 μM in a 96-well plate format. The reaction mix contained 0.1 μM Pks13-TE in 0.1 M Tris-HCl, pH 7 buffer with 1 μl of each dilution of the compound or DMSO in a total volume of 99 μl. The reaction was initiated by addition of 1 μl of 2 mM 4-MUH in DMSO (20 μM final concentration) to the reaction mix. Initial velocity data was obtained by monitoring increase in the fluorescence due to hydrolysis of the substrate using PolarStar Omega plate reader at 10 min intervals over 60 min. The data points were collected in triplicate and the averaged value was used to generate concentration-response plots for the test compound and its analogs. The IC₅₀ value for each compound was obtained by nonlinear regression curve fitting of a four-parameter variable slope equation to the dose-response data using Prism software. The IC₅₀ values of the test compound for Pks13-TE mutants were determined in the same way as that for wt Pks13-TE, however, the testing concentration of the test compound ranged from 0.04 to 40 μM and the substrate 4-MUH was used at a final concentration of 20 μM in the reaction mixture.

Whole Cell and Cytotoxicity Testing

Whole cell testing for determining MIC was done using Alamarblue assay in 96-well plates. Mtb mc^{g}-7000 strain cells were grown in 7H9 media supplemented with OADC (Middlebrook), 0.05% Tyloxapol (Sigma), and 25 mg/ml pantothenate to an OD₆₀₀ of 1-2. The cells were then diluted into testing media (7H9 media with 0.2% dextrose, 0.085% NaCl, 0.05% Tyloxapol, and 25 mg/ml pantothenate) to an OD₆₀₀ of 0.01 and dispensed into testing plates at 200 μl per well. Then the compounds were added as a 2-fold serial dilution in DMSO (2% DMSO final in each well). The test plates also had a DMSO only control and a Rifampicin control. The plates were incubated with shaking at 37° C. for 6 days and then stained with resazurin (Sigma) for an additional 2 days at 37° C. After staining the fluorescence of reduced resazurin was read (λ_{Ex}=544 nm, λ_{Em}=590 nm) using PolarStar Omega plate reader. The fluorescence data were plotted as percent growth inhibition against the compound concentration and curve fitting was done by nonlinear regression using Prism software. Minimum inhibitory concentration (MIC) values were determined from the fitted curves.

Compounds were tested for toxicity by the Human Dermal Fibroblast (HDF) cytotoxicity assay. HDF cells were purchased from ATCC (Manassas, Va.). The cells were cultured in DMEM (Lonza) media supplemented with 10% fetal bovine serum (Lonza) and penicillin/streptomycin (Lonza). For setting the cytotoxicity assay, compound stocks were serially diluted in phosphate buffered saline (PBS) plus 10% DMSO. On the day of assay, HDF cells were trypsinized, counted and resuspended at a concentration of 64,000 cells/ml in the media. Cells were plated, overlaid with the compound serial dilutions and incubated at 37° C. After 48 h, resazurin dye was added and the assay plates were cultured for another 24 h. The next day the absorbance of the resazurin was measured on a microplate reader (BMG Labtech) to assess cell death. Cytotoxicity was determined as a percent of dead cells versus living cells.

In Vivo Efficacy and Safety

6-8 week old Balb/C female mice from Charles River were rested at least one week prior to Mtb infection with ˜50-100 bacilli/mouse (Erdman lux strain lot #11/20/08) by Low Dose Aerosol administration via Glas-Col Inhalation Exposure System. Whole lungs and spleens were extracted and homogenized in PBS. CFU was determined by counting after plating homogenates on 7H-11 agar plates and incubating in a 37° C. dry-air incubator for ˜3 weeks. Therapy, administered via oral gavage, was started on day 27 post-infection and continued for 4 weeks. Drugs were administered daily, for 5 days a week, by oral gavage in a volume of 200 μl/animal in canola oil. After one month of treatment; 5-6 mice from each group (untreated and drug treated mice) were sacrificed and bacterial loads were determined. Plating of lung and spleen homogenate was conducted as described above.

While numerous changes may be made by those skilled in the art, such changes are encompassed within the spirit of this invention as illustrated, in part, by the appended claims.

What is claimed is:

1. A composition comprising at least one benzofuran derivative having the following formula: 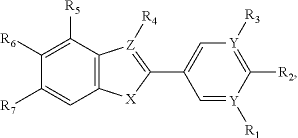
wherein:
X is O or NH;
each Y group is independently selected from C and N;
Z is C or N;
R₁, R₂ and R₃ are independently selected from hydrogen, methoxy, hydroxyl, fluoro, nitrile, and carboxamide moieties;
R₄ is selected from the group consisting of CH₂OH, COOEt, COOH, CONHMe, CONHEt, and amides of cyclic and acyclic secondary or tertiary amines;
R₅ is an alkyl, cyclic alkyl, or heterocyclic alkyl group, optionally substituted with substituents selected from hydroxyl, alkoxy, halogen, amine, alkylamine, hydroxyalkylamine, dialkylamine, dialkylaminealkyl, carboxy, carboxamide, acylamine, sulfoxide, sulfone, aryl, heteroaryl, and heterocyclic groups;
R₆ is selected from H, OMe, and OH; and
R₇ is selected from H, NO₂, NH₂, and NHAc.

2. The composition of claim 1, wherein the at least one benzofuran derivative is not:

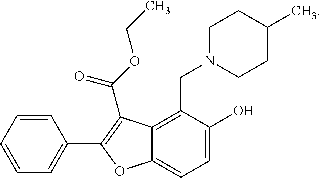 [SMILES: CCOC(=O)c1c(-c2ccccc2)oc2ccc(O)c(CN3CCC(C)CC3)c12]

3. The composition of claim 1, wherein the R₂ substituent of the at least one benzofuran derivative is a hydroxyl moiety.

4. The composition of claim 1, wherein the R₄ substituent of the at least one benzofuran derivative is a CONHMe moiety.

5. The composition of claim 1, wherein the at least one benzofuran derivative has a half-maximal inhibitory concentration against wild-type Pks13 thioesterase of 0.25 μM or less.

6. The composition of claim 1, wherein the at least one benzofuran derivative has a minimum inhibitory concentration against Mycobacterium tuberculosis bacilli of 2 μM or less.

7. The composition of claim 1, wherein the at least one benzofuran derivative is selected from the group consisting of Inhibitor 31 and Inhibitor 32.

8. A method of inhibiting a bacterium in a patient comprising administering to the patient, in an amount effective to inhibit the bacterium, a composition comprising at least one benzofuran derivative having the following formula: 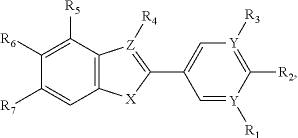
wherein:
X is O or NH;
each Y group is independently selected from C and N;
Z is C or N;
R₂ and R₃ are independently selected from hydrogen, methoxy, hydroxyl, fluoro, nitrile, and carboxamide moieties;
R₄ is selected from the group consisting of CH₂OH, COOEt, COOH, CONHMe, CONHEt, and amides of cyclic and acyclic secondary or tertiary amines;
R₅ is an alkyl, cyclic alkyl, or heterocyclic alkyl group, optionally substituted with substituents selected from hydroxyl, alkoxy, halogen, amine, alkylamine, hydroxyalkylamine, dialkylamine, dialkylaminealkyl, carboxy, carboxamide, acylamine, sulfoxide, sulfone, aryl, heteroaryl, and heterocyclic groups;
R₆ is selected from H, OMe, and OH; and
R₇ is selected from H, NO₂, NH₂, and NHAc.

9. The method of claim 8, wherein the bacterium is a Mycobacterium.

10. The method of claim 9, wherein the Mycobacterium is Mycobacterium tuberculosis.

11. The method of claim 8, further comprising administering to the patient at least one additional antibiotic drug.

12. The method of claim 11, wherein the at least one additional antibiotic drug is selected from the group consisting of with one or more drugs selected from the group consisting of isoniazid, rifampicin, pyrazinamide, ethambutol, rifapentine, rifabutin, streptomycin, kanamycin, and amikacin, capreomycin, viomycin, ciprofloxacin, levofloxacin, moxifloxacin, ofloxacin, gatifloxacin, para-aminosalicylic acid, cycloserine, terizidone, ethionamide, prothionamide, thioacetazone, linezolid, clofazimine, amoxicillin, clavulanate, imipenem, cilastatin, and clarithromycin.

13. The composition of claim 1, wherein the R₂ substituent of the at least one benzofuran derivative is a hydroxyl moiety and the R₄ substituent of the at least one benzofuran derivative is a CONHMe moiety.

14. The composition of claim 13, wherein the at least one benzofuran derivative has a half-maximal inhibitory concentration against wild-type Pks13 thioesterase of 0.25 μM or less.

15. The composition of claim 13, wherein the at least one benzofuran derivative has a minimum inhibitory concentration against Mycobacterium tuberculosis bacilli of 2 μM or less.

16. The method of claim 8, wherein the R₂ substituent of the at least one benzofuran derivative is a hydroxyl moiety and the R₄ substituent of the at least one benzofuran derivative is a CONHMe moiety.

17. The method of claim 16, wherein the at least one benzofuran derivative has a half-maximal inhibitory concentration against wild-type Pks13 thioesterase of 0.25 μM or less.

18. The method of claim 16, wherein the at least one benzofuran derivative has a minimum inhibitory concentration against Mycobacterium tuberculosis bacilli of 2 μM or less.
